(19) United States
(12) Patent Application Publication
(10) Pub. No.: US 2020/0107925 A1
(43) Pub. Date: Apr. 9, 2020

(54) STENT-GRAFTS FOR SEALING AROUND EXTERNAL DISTURBANCES

(71) Applicant: ENDOSPAN LTD., Herzilyia, Pituach (IL)

(72) Inventors: Or ZIGELBOIM, Ness Ziona (IL); Yaniv MARMUR, Yokneam Moshava (IL); Alon SHALEV, Ra'anana (IL)

(73) Assignee: ENDOSPAN LTD., Herzilyia, Pituach (IL)

(21) Appl. No.: 16/495,670

(22) PCT Filed: Mar. 21, 2018
(86) PCT No.: PCT/IL2018/050325

§ 371 Date: Sep. 19, 2019

Related U.S. Application Data
(60) Provisional application No. 62/474,391, filed on Mar. 21, 2017.

(51) Int. Cl. A61F 2/07; A61F 2/89

(52) U.S. Cl. CPC A61F 2/07; A61F 2250/0039; A61F 2220/0008; A61F 2/89

(57) ABSTRACT

An endovascular stent-graft (10, 110, 210, 310) is provided that includes elastic struts (20) and a fluid flow guide (30) that is fixed to first and second subsets (32, 34) of the struts (20). The struts (20) of the first subset (32) are arranged as circumferential cells (80) in circumferentially-continuous rings (56), which cause the fluid flow guide (30) to define substantially cylindrical tubular portions (40). The struts (20) of the second subset (34) cause the fluid flow guide (30) to define a bulge (42) having a greatest bulge radius from a central longitudinal axis (38) that is at least 5% greater than an average radius of the substantially cylindrical tubular portions (40) proximally and distally adjacent the bulge (42). The struts (20) of the second subset (34) define tip portions (50). The number of the tip portions (50) of the struts (20) of the second subset (34) that define the bulge (42) is at least 50% greater than the average number of circumferential cells (80) in the two circumferentially-continuous rings (56) proximally and distally adjacent the bulge (42).

### CROSS-REFERENCE TO RELATED APPLICATIONS

The present application claims priority from U.S. Provisional Application 62/474,391, filed Mar. 21, 2017, which is assigned to the assignee of the present application and is incorporated herein by reference.

### FIELD OF THE APPLICATION

The present invention relates generally to implantable medical devices, and specifically to implantable stent-grafts.

### BACKGROUND OF THE APPLICATION

Endovascular prostheses are sometimes used to treat aortic aneurysms. Such treatment includes implanting a stent or stent-graft within the diseased vessel to bypass the anomaly. An aneurysm is a sac formed by the dilation of the wall of the artery. Aneurysms may be congenital, but are usually caused by disease or, occasionally, by trauma. Aortic aneurysms which commonly form between the renal arteries and the iliac arteries are referred to as abdominal aortic aneurysms (“AAAs”). Other aneurysms occur in the aorta, such as thoracic aortic aneurysms (“TAAs”) and aortic uni-iliac (“AUI”) aneurysms. A TAA may occur downstream the aortic arch, i.e., in the descending aorta. Alternatively, a TAA may occur in the aortic arch itself, where the aorta branches to supply the brachiocephalic, left carotid and subclavian arteries, or may occur in the ascending aorta.

Endo-Vascular Aneurysm Repair (EVAR) has transformed the practice of treatment of aortic aneurysms from an open surgical approach to a much less invasive surgical approach. The first step of an endovascular intervention usually requires introducing a delivery system into the vasculature of a subject. If the crossing profile, i.e., the external diameter, of the delivery system is 24 Fr or lower (3 Fr=1 millimeter), a true percutaneous approach may be used, because vascular closure devices are available for proper closure of such puncture sites.

Blood vessels occasionally weaken or even rupture. For example, in the aortic artery, the vascular wall can weaken or tear, resulting in dangerous conditions such as aneurysm and dissection. Treatment of such conditions can be performed by implanting a stent-graft within the vascular system using minimally-invasive surgical procedures. An endoluminal stent-graft typically includes one or more stents affixed to graft material and is delivered to the treatment site by endovascular insertion. Once the endoluminal stent-graft is radially enlarged, it should remain in place indefinitely by self-attachment to the vessel wall, acting as a substitute vessel for the flow of blood or other fluids.

Aortic dissection is a tear or partial tear in the inner wall of the aorta, which causes blood to flow between the layers of the wall of the aorta, forcing the layers apart. Aortic dissections may be divided into two types in accordance with the Stanford classification. Type A dissections involve the ascending aorta and/or aortic arch, and possibly the descending aorta. Type B dissections involve the descending aorta or the arch (distal to right brachiocephalic artery origin), without involvement of the ascending aorta.

### SUMMARY OF THE APPLICATION

Embodiments of the present invention provide an endovascular stent-graft, which comprises struts and a fluid flow guide, which is fixed to first and second subsets of the struts. The struts of the first and the second subsets are elastic such that, when the stent-graft assumes the radially-expanded state:
the fluid flow guide defines a lumen having a central longitudinal axis,
the struts of the first subset cause the fluid flow guide to define a plurality of substantially cylindrical tubular portions, and
the struts of the second subset cause the fluid flow guide to define a bulge having a greatest bulge radius from the central longitudinal axis, which greatest bulge radius is at least 5% greater than an average radius of the substantially cylindrical tubular portions proximally and distally adjacent the bulge.

For some applications, the bulge is configured to reduce the likelihood of long-term leakage (i.e., blood flow) through gutters, i.e., the residual intravascular space disposed outside the lumens of the endovascular stent-graft and branching stent-grafts disposed alongside the endovascular stent-graft. Alternatively or additionally, the bulge may reduce the likelihood of blood flow between the endovascular stent-graft and features of the anatomy of the blood vessel wall, such as isolated regions of plaque, calcifications, or thrombus, all of which alter the circularity of the blood vessel wall and might otherwise present issues for good sealing between the stent-graft and the blood vessel wall. As a result, the likelihood of type 1 endoleak is reduced.

For some applications, the struts of the second subset have an average wall thickness, measured radially, that is no more than 80% (e.g., no more than 60%) of an average wall thickness of the struts of the first subset, measured radially. Such a lower wall thickness may contribute to a lower spring constant of the struts of the second subset than the struts of the first subset, which may facilitate more local deformation (indentation) of the bulge around a branching stent-graft, without unnecessarily crushing the branching stent-graft, or necessitating internally reinforcement of the branching stent-graft with additional metallic stents.

For some applications, the struts of the first subset are arranged as a plurality of circumferential cells in circumferentially-continuous rings, which cause the fluid flow guide to define the plurality of substantially cylindrical tubular portions. For some applications, the struts of the second subset define tip portions. For some of these applications, the number of tip portions of the struts of the second subset that define the bulge is at least 30% (typically at least 50%) greater than the average number of the circumferential cells in the two the circumferentially-continuous rings proximally and distally adjacent the bulge, such as least 175% greater, no more than 250% greater, and/or between 175% and 250% (e.g., 200%) of the average number of the circumferential cells. Providing such a relatively large number of tip portions may facilitate more local deformation (indentation) of the bulge around a branching stent-graft, without unnecessarily crushing the branching stent-graft, or necessitating internally reinforcement of the branching stent-graft with additional metallic stents.

There is therefore provided, in accordance with an Inventive concept 1 of the present invention, apparatus for use with a delivery catheter, the apparatus including an endovascular stent-graft, which is configured to initially be positioned in the delivery catheter in a radially-compressed state, and to assume a radially-expanded state upon being deployed from the delivery catheter, and which includes:

struts; and

a fluid flow guide, which includes at least one biologically-compatible substantially blood-impervious fabric, and which is fixed to first and second subsets of the struts, wherein the first and the second subsets do not include any common struts,

wherein the struts of the first and the second subsets are elastic such that, when the stent-graft assumes the radially-expanded state:
the fluid flow guide defines a lumen having a central longitudinal axis,
the struts of the first subset are arranged as a plurality of circumferential cells in circumferentially-continuous rings, which cause the fluid flow guide to define a plurality of substantially cylindrical tubular portions,
the struts of the second subset cause the fluid flow guide to define a bulge having a greatest bulge radius from the central longitudinal axis, which greatest bulge radius is at least 5% greater than an average radius of the substantially cylindrical tubular portions proximally and distally adjacent the bulge,
the struts of the second subset define tip portions, and
the number of the tip portions of the struts of the second subset that define the bulge is at least 50% greater than the average number of circumferential cells in the two circumferentially-continuous rings proximally and distally adjacent the bulge.
   Inventive concept 2. The apparatus according to Inventive concept 1, wherein the number of the tip portions of the struts of the second subset that define the bulge equals between 175% and 250% of the average number of circumferential cells.
   Inventive concept 3. The apparatus according to Inventive concept 2, wherein the number of the tip portions of the struts of the second subset that define the bulge equals 200% of the average number of circumferential cells.
   Inventive concept 4. The apparatus according to Inventive concept 1, wherein the number of the tip portions of the struts of the second subset that define the bulge equals between 12 and 16.
   Inventive concept 5. The apparatus according to Inventive concept 1, wherein the bulge completely circumferentially encircles the stent-graft.
   Inventive concept 6. The apparatus according to Inventive concept 1, wherein the tip portions are directly sutured to the fluid flow guide.
   Inventive concept 7. The apparatus according to Inventive concept 1, wherein the tip portions are shaped so as to define respective atraumatic features.
   Inventive concept 8. The apparatus according to Inventive concept 1, the fluid flow guide is arranged such that all of the fabric defines the lumen when the stent-graft assumes the radially-expanded state.
   Inventive concept 9. The apparatus according to Inventive concept 1, wherein the circumferentially-continuous rings undulate, such that the circumferential cells are shaped so as to define respective peaks and troughs.
   Inventive concept 10. The apparatus according to Inventive concept 9, wherein the peaks and troughs are substantially semicircular, have a first average radius of curvature, and are shaped so as to define respective strain-distribution features having a second average radius of curvature equal to between 10% and 50% of the first average radius of curvature.
   Inventive concept 11. The apparatus according to Inventive concept 1, wherein an axial length of the bulge equals between 10% and 40% of a difference between (a) the greatest bulge radius and (b) the average radius of the substantially cylindrical tubular portions proximally and distally adjacent the bulge.
   Inventive concept 12. The apparatus according to Inventive concept 1, wherein the struts of the first and the second subsets are arranged and shaped such that the struts of the first subset apply a radially-outward force that is at least 20% greater than a radially-outward applied by the struts of the second subset.
   Inventive concept 13. The apparatus according to Inventive concept 12, wherein the struts of the first and the second subsets are arranged and shaped such that the struts of the first subset apply the radially-outward force that is at least 40% greater than the radially-outward applied by the struts of the second subset.
   Inventive concept 14. The apparatus according to Inventive concept 1, wherein the circumferential cells are W-shaped.
   Inventive concept 15. The apparatus according to Inventive concept 1, wherein the struts of the first and the second subsets are superelastic.
   Inventive concept 16. The apparatus according to Inventive concept 1, wherein the circumferential cells are diamond-shaped.
   Inventive concept 17. The apparatus according to any one of Inventive concepts 1-16,

wherein the bulge is a first bulge,

wherein the fluid flow guide is fixed to the first subset of the struts, the second subset of the struts, and a third subset of the struts so as to define the lumen, wherein the first, the second, and the third subsets do not include any common struts, and

wherein the struts of the third subset are elastic such that, when the stent-graft assumes the radially-expanded state:
the struts of the third subset cause the fluid flow guide to define a second bulge having a greatest bulge radius from the central longitudinal axis, which greatest bulge radius is at least 5% greater than an average radius of the substantially cylindrical tubular portions proximally and distally adjacent the second bulge,
the struts of the third subset define tip portions, and
the number of the tip portions of the struts of the third subset that define the second bulge is at least 50% greater than the average number of circumferential cells in the two circumferentially-continuous rings proximally and distally adjacent the second bulge.
   Inventive concept 18. The apparatus according to any one of Inventive concepts 1-16, wherein at least a first one of the struts of the second subset is structurally integral with at least a second one of the struts of the first subset.
   Inventive concept 19. The apparatus according to Inventive concept 18,

wherein all of the struts of the second subset are structurally integral with at least one of the struts of the first subset,

wherein none of the struts of the second subset is directly connected to any of the other struts of the second subset, and

wherein none of the struts of the second subset is indirectly connected to any of the other struts of the second subset by any struts other than the struts of the first subset.

Inventive concept 20. The apparatus according to Inventive concept 18, wherein, for at least a first one of the tip portions of first one of the struts of the second subset, an angle is defined by (a) the central longitudinal axis and (b) a line defined by (i) the first one of the tip portions and (ii) a junction between the first one of the struts of the second subset and the second one of the struts of the first subset, and the angle is between 5 and 60 degrees.
Inventive concept 21. The apparatus according to Inventive concept 20, wherein the angle is between 10 and 30 degrees.
Inventive concept 22. The apparatus according to any one of Inventive concepts 1-16,

wherein the stent-graft further includes sutures that secure the struts of the first and the second subsets to the fluid flow guide, and

wherein, for at least a first one of the tip portions of the first one of the struts of the second subset
the first one of the struts of the second subset has a length measured along the first one of the struts between (a) the first tip portion and (b) a junction between the first one of the struts of the second subset and the second one of the struts of the first subset,
a far half of the first one of the struts extends from the first tip portion along 50% of the length of the first one of the struts of the second subset, and
none of the sutures are disposed along at least 50% of the far half of the first one of the struts of the second subset.
   Inventive concept 23. The apparatus according to any one of Inventive concepts 1-16, wherein the struts of the first subset are arranged in a plurality of undulating circumferentially-continuous rings having alternating peaks and troughs, and wherein a first plurality of the struts of the second subset originate in a proximal half of one of the undulating circumferentially-continuous rings, and a second plurality of the struts of the second subset originate in a distal half of the one of the undulating circumferentially-continuous rings.
   Inventive concept 24. The apparatus according to Inventive concept 23, wherein the first plurality of the struts of the second subset originate in a proximal 20% of an axial height of the one of the undulating circumferentially-continuous rings.
   Inventive concept 25. The apparatus according to Inventive concept 24, wherein the first plurality of the struts of the second subset originate at proximal-most sites of the one of the undulating circumferentially-continuous rings.
   Inventive concept 26. The apparatus according to Inventive concept 24, wherein the second plurality of the struts of the second subset originate in a distal 20% of the axial height of the one of the undulating circumferentially-continuous rings.
   Inventive concept 27. The apparatus according to Inventive concept 26,

wherein the first plurality of the struts of the second subset originate at proximal-most sites of the one of the undulating circumferentially-continuous rings, and

wherein the second plurality of the struts of the second subset originate at distal-most sites of the one of the undulating circumferentially-continuous rings.

Inventive concept 28. The apparatus according to Inventive concept 23, wherein a first plurality of the struts of the second subset originate at two or more axially different locations of one of the undulating circumferentially-continuous rings.
Inventive concept 29. The apparatus according to any one of Inventive concepts 1-16, wherein at least one of the struts of the second subset is not structurally integral with any of the struts of the first subset.
Inventive concept 30. The apparatus according to any one of Inventive concepts 1-16,

wherein the fluid flow guide is fixed to the first subset of the struts, the second subset of the struts, and a third subset of the struts so as to define the lumen, wherein the first, the second, and the third subsets do not include any common struts, and

wherein the struts of the third subset are elastic such that, when the stent-graft assumes the radially-expanded state, the struts of the third subset cause the fluid flow guide to define a flared axial portion that extends to one end of the fluid flow guide, the flared axial portion having (a) a greatest flared radius from the central longitudinal axis, which greatest flared radius is at least 5% greater than a radius of the substantially cylindrical tubular portion axially adjacent the flared axial portion, and (b) an axial length equal to between 5% and 20% of a difference between (i) the greatest flared radius and (ii) the radius of the substantially cylindrical tubular portion axially adjacent the flared axial portion.

Inventive concept 31. The apparatus according to Inventive concept 30,

wherein the flared axial portion is a first flared axial portion, and the one end of the fluid flow guide is a first end of the fluid flow guide, and

wherein the struts of the third subset are elastic such that, when the stent-graft assumes the radially-expanded state, the struts of the third subset cause the fluid flow guide to additionally define a second flared axial portion that extends to a second end of the fluid flow guide, the second flared axial portion having (a) a greatest flared radius from the central longitudinal axis, which greatest flared radius is at least 5% greater than an average radius of the substantially cylindrical tubular portion axially adjacent the second flared axial portion, and (b) an axial length equal to between 5% and 20% of a difference between (i) the greatest flared radius and (ii) the radius of the substantially cylindrical tubular portion axially adjacent the second flared axial portion.

Inventive concept 32. The apparatus according to any one of Inventive concepts 1-16,

wherein the stent-graft further includes sutures that secure the struts of the first and the second subsets to the fluid flow guide, and

wherein at least 80% of a surface area of the struts of the first subset is within 3 mm of at least one of the sutures that secure the struts of the first subset to the fluid flow guide.

Inventive concept 33. The apparatus according to Inventive concept 32, wherein no more than 50% of the surface area of the struts of the second subset is within 3 mm of at least one of the sutures that secure the struts of the second subset to the fluid flow guide.
Inventive concept 34. The apparatus according to any one of Inventive concepts 1-16, wherein respective circumferences of all substantially cylindrical tubular portions of the fluid flow guide vary by less than 10%.
Inventive concept 35. The apparatus according to any one of Inventive concepts 1-16, wherein a circumference of a first one of the substantially cylindrical tubular portions is at least 10% greater than a circumference of a second one of the substantially cylindrical tubular portions.
Inventive concept 36. The apparatus according to any one of Inventive concepts 1-16, wherein when the endovascular stent-graft is removably disposed in the delivery catheter in the radially-compressed state, the struts of the first subset do not coincide with the struts of the second subset.
Inventive concept 37. The apparatus according to any one of Inventive concepts 1-16,

wherein the stent-graft is a main stent-graft,

wherein the fluid flow guide is not shaped so as to define any fenestrations or scallops, and

wherein the apparatus further includes one or more branching stent-grafts.

Inventive concept 38. The apparatus according to any one of Inventive concepts 1-16,

wherein the stent-graft is a main stent-graft,

wherein the fluid flow guide is not shaped so as to define one or more openings selected from the group of openings consisting of: fenestrations, scallops, and fenestrations and scallops, and

wherein the apparatus further includes a number of branching stent-grafts, the number greater than a number of the openings.

There is further provided, in accordance with an Inventive concept 39 of the present invention, apparatus for use with a delivery catheter, the apparatus including an endovascular stent-graft, which is configured to initially be positioned in the delivery catheter in a radially-compressed state, and to assume a radially-expanded state upon being deployed from the delivery catheter, and which includes:

struts; and

a fluid flow guide, which includes at least one biologically-compatible substantially blood-impervious fabric, and which is fixed to first and second subsets of the struts, wherein the first and the second subsets do not include any common struts,

wherein the struts of the first and the second subsets are elastic such that, when the stent-graft assumes the radially-expanded state:
the fluid flow guide defines a lumen having a central longitudinal axis,
the struts of the first subset are arranged as a plurality of circumferential cells in circumferentially-continuous rings, which cause the fluid flow guide to define a plurality of substantially cylindrical tubular portions,
the struts of the second subset are arranged as a plurality of bulge-inducing units that cause the fluid flow guide to define a bulge having a greatest bulge radius from the central longitudinal axis, which greatest bulge radius is at least 5% greater than an average radius of the substantially cylindrical tubular portions proximally and distally adjacent the bulge, and
an average circumferential width of the bulge-inducing units is no more than 25% of an average circumferential width of the circumferential cells in the two circumferentially-continuous rings proximally and distally adjacent the bulge.
   Inventive concept 40. The apparatus according to Inventive concept 39, wherein the average circumferential width of the bulge-inducing units is no more than 20% of the average circumferential width of the circumferential cells.
   Inventive concept 41. The apparatus according to Inventive concept 40, wherein the average circumferential width of the bulge-inducing units is no more than 10% of the average circumferential width of the circumferential cells.
   Inventive concept 42. The apparatus according to Inventive concept 39, wherein at least one of the bulge-inducing units includes exactly one of the struts of the second subset.
   Inventive concept 43. The apparatus according to Inventive concept 39, wherein at least one of the bulge-inducing units includes two of the struts of the second subset.
   Inventive concept 44. The apparatus according to Inventive concept 39, wherein the number of the bulge-inducing units is at least 50% greater than the average number of circumferential cells in the two circumferentially-continuous rings proximally and distally adjacent the bulge.
   Inventive concept 45. The apparatus according to Inventive concept 44, wherein the number of the bulge-inducing units equals between 175% and 250% of the average number of circumferential cells.
   Inventive concept 46. The apparatus according to Inventive concept 45, wherein the number of the bulge-inducing units equals 200% of the average number of circumferential cells.
   Inventive concept 47. The apparatus according to Inventive concept 39, wherein the circumferentially-continuous rings undulate, such that the circumferential cells are shaped so as to define respective peaks and troughs.
   Inventive concept 48. The apparatus according to Inventive concept 39, wherein the circumferential cells are W-shaped.
   Inventive concept 49. The apparatus according to Inventive concept 39, wherein the circumferential cells are diamond-shaped.
   Inventive concept 50. The apparatus according to Inventive concept 39, wherein an axial length of the bulge equals between 10% and 40% of a difference between (a) the greatest bulge radius and (b) the average radius of the substantially cylindrical tubular portions proximally and distally adjacent the bulge.
   Inventive concept 51. The apparatus according to Inventive concept 39, wherein the bulge completely circumferentially encircles the stent-graft.
   Inventive concept 52. The apparatus according to Inventive concept 39, wherein the struts of the first and the second subsets are superelastic.
   Inventive concept 53. The apparatus according to any one of Inventive concepts 39-52, wherein the stent-graft is a main stent-graft, wherein the fluid flow guide is not shaped so as to define any fenestrations or scallops, and wherein the apparatus further includes one or more branching stent-grafts.
   Inventive concept 54. The apparatus according to any one of Inventive concepts 39-52,

wherein the stent-graft is a main stent-graft,

wherein the fluid flow guide is not shaped so as to define one or more openings selected from the group of openings consisting of: fenestrations, scallops, and

fenestrations and scallops, and

wherein the apparatus further includes a number of branching stent-grafts, the number greater than a number of the openings.

Inventive concept 55. The apparatus according to any one of Inventive concepts 39-52, wherein the struts of the second subset have an average wall thickness, measured radially, that is no more than 80% of an average wall thickness of the struts of the first subset, measured radially.
Inventive concept 56. The apparatus according to Inventive concept 55, wherein the average wall thickness of the struts of the second subset, measured radially, is no more than 60% of the average wall thickness of the struts of the first subset, measured radially.
Inventive concept 57. The apparatus according to any one of Inventive concepts 39-52,

wherein the fluid flow guide is fixed to the first subset of the struts, the second subset of the struts, and a third subset of the struts so as to define the lumen, wherein the first, the second, and the third subsets do not include any common struts, and

wherein the struts of the third subset are elastic such that, when the stent-graft assumes the radially-expanded state, the struts of the third subset cause the fluid flow guide to define a flared axial portion that extends to one end of the fluid flow guide, the flared axial portion having (a) a greatest flared radius from the central longitudinal axis, which greatest flared radius is at least 5% greater than a radius of the substantially cylindrical tubular portion axially adjacent the flared axial portion, and (b) an axial length equal to between 5% and 20% of a difference between (i) the greatest flared radius and (ii) the radius of the substantially cylindrical tubular portion axially adjacent the flared axial portion.

Inventive concept 58. The apparatus according to Inventive concept 57,

wherein the flared axial portion is a first flared axial portion, and the one end of the fluid flow guide is a first end of the fluid flow guide, and

wherein the struts of the third subset are elastic such that, when the stent-graft assumes the radially-expanded state, the struts of the third subset cause the fluid flow guide to additionally define a second flared axial portion that extends to a second end of the fluid flow guide, the second flared axial portion having (a) a greatest flared radius from the central longitudinal axis, which greatest flared radius is at least 5% greater than an average radius of the substantially cylindrical tubular portion axially adjacent the second flared axial portion, and (b) an axial length equal to between 5% and 20% of a difference between (i) the greatest flared radius and (ii) the radius of the substantially cylindrical tubular portion axially adjacent the second flared axial portion.

There is still further provided, in accordance with an Inventive concept 59 of the present invention, apparatus for use with a delivery catheter, the apparatus including an endovascular stent-graft, which is configured to initially be positioned in the delivery catheter in a radially-compressed state, and to assume a radially-expanded state upon being deployed from the delivery catheter, and which includes:

struts; and

a fluid flow guide, which includes at least one biologically-compatible substantially blood-impervious fabric, and which is fixed to first and second subsets of the struts, wherein the first and the second subsets do not include any common struts, and wherein the struts of the second subset have an average wall thickness, measured radially, that is no more than 80% of an average wall thickness of the struts of the first subset, measured radially,

wherein the struts of the first and the second subsets are elastic such that, when the stent-graft assumes the radially-expanded state:
the fluid flow guide defines a lumen having a central longitudinal axis,
the struts of the first subset cause the fluid flow guide to define a plurality of substantially cylindrical tubular portions, and
the struts of the second subset cause the fluid flow guide to define a bulge having a greatest bulge radius from the central longitudinal axis, which greatest bulge radius is at least 5% greater than an average radius of the substantially cylindrical tubular portions proximally and distally adjacent the bulge.
   Inventive concept 60. The apparatus according to Inventive concept 59, wherein the average wall thickness of the struts of the second subset, measured radially, is no more than 60% of the average wall thickness of the struts of the first subset, measured radially.
   Inventive concept 61. The apparatus according to Inventive concept 59,

wherein the struts of the first subset have a first average cross-sectional area, measured perpendicular to respective axes of the struts of the first subset,

wherein the struts of the second subset have a second average cross-sectional area, measured perpendicular to respective axes of the struts of the second subset, and

wherein the second average cross-sectional area is no more than 80% of the first cross-sectional area.

Inventive concept 62. The apparatus according to Inventive concept 59, the fluid flow guide is arranged such that all of the fabric defines the lumen when the stent-graft assumes the radially-expanded state.
Inventive concept 63. The apparatus according to Inventive concept 59,

wherein the bulge is a first bulge,

wherein the fluid flow guide is fixed to the first subset of the struts, the second subset of the struts, and a third subset of the struts so as to define the lumen, wherein the first, the second, and the third subsets do not include any common struts, and wherein the struts of the third subset have an average wall thickness, measured radially, that is no more than 80% of the average wall thickness of the struts of the first subset, measured radially, and

wherein the struts of the third subset are elastic such that, when the stent-graft assumes the radially-expanded state, the struts of the third subset cause the fluid flow guide to define a second bulge having a greatest bulge radius from the central longitudinal axis, which greatest bulge radius is at least 5% greater than an average radius of the substantially cylindrical tubular portions proximally and distally adjacent the second bulge.

Inventive concept 64. The apparatus according to Inventive concept 59, wherein the struts of the first and the second subsets are arranged and shaped such that the struts of the first subset apply a radially-outward force that is at least 20% greater than a radially-outward applied by the struts of the second subset.
Inventive concept 65. The apparatus according to Inventive concept 64, wherein the struts of the first and the second subsets are arranged and shaped such that the struts of the first subset apply the radially-outward force that is at least 40% greater than the radially-outward applied by the struts of the second subset.
Inventive concept 66. The apparatus according to Inventive concept 59, wherein the struts of the first and the second subsets are superelastic.
Inventive concept 67. The apparatus according to Inventive concept 59, wherein the bulge completely circumferentially encircles the stent-graft.
Inventive concept 68. The apparatus according to any one of Inventive concepts 59-67, wherein at least a first one of the struts of the second subset is structurally integral with at least a second one of the struts of the first subset.
Inventive concept 69. The apparatus according to Inventive concept 68,

wherein all of the struts of the second subset are structurally integral with at least one of the struts of the first subset,

wherein none of the struts of the second subset is directly connected to any of the other struts of the second subset, and

wherein none of the struts of the second subset is indirectly connected to any of the other struts of the second subset by any struts other than the struts of the first subset.

Inventive concept 70. The apparatus according to Inventive concept 68,

wherein the first one of the struts of the second subset defines a first tip portion, and

wherein an angle is defined by (a) the central longitudinal axis and (b) a line defined by (i) the first tip portion and (ii) a junction between the first one of the struts of the second subset and the second one of the struts of the first subset, and the angle is between 5 and 60 degrees.

Inventive concept 71. The apparatus according to Inventive concept 70, wherein the angle is between 10 and 30 degrees.
Inventive concept 72. The apparatus according to Inventive concept 68,

wherein the first one of the struts of the second subset defines a first tip portion,

wherein the first one of the struts of the second subset has a length measured along the first one of the struts of the second subset between (a) the first tip portion and (b) a junction between the first one of the struts of the second subset and the second one of the struts of the first subset,

wherein a far half of the first one of the struts of the second subset extends from the first tip portion along 50% of the length of the first one of the struts of the second subset,

wherein the stent-graft further includes sutures that secure the struts of the first and the second subsets to the fluid flow guide, and

wherein none of the sutures are disposed along at least 50% of the far half of the first one of the struts of the second subset.

Inventive concept 73. The apparatus according to any one of Inventive concepts 59-67,

wherein the struts of the first subset are arranged in a plurality of undulating circumferentially-continuous rings having alternating peaks and troughs, and

wherein a first plurality of the struts of the second subset originate in a proximal half of one of the undulating circumferentially-continuous rings, and a second plurality of the struts of the second subset originate in a distal half of the one of the undulating circumferentially-continuous rings.

Inventive concept 74. The apparatus according to Inventive concept 73, wherein the first plurality of the struts of the second subset originate in a proximal 20% of an axial height of the one of the undulating circumferentially-continuous rings.
Inventive concept 75. The apparatus according to Inventive concept 74, wherein the first plurality of the struts of the second subset originate at proximal-most sites of the one of the undulating circumferentially-continuous rings.
Inventive concept 76. The apparatus according to Inventive concept 74, wherein the second plurality of the struts of the second subset originate in a distal 20% of the axial height of the one of the undulating circumferentially-continuous rings.
Inventive concept 77. The apparatus according to Inventive concept 76,

wherein the first plurality of the struts of the second subset originate at proximal-most sites of the one of the undulating circumferentially-continuous rings, and

wherein the second plurality of the struts of the second subset originate at distal-most sites of the one of the undulating circumferentially-continuous rings.

Inventive concept 78. The apparatus according to Inventive concept 73, wherein a first plurality of the struts of the second subset originate at two or more axially different locations of one of the undulating circumferentially-continuous rings.
Inventive concept 79. The apparatus according to any one of Inventive concepts 59-67, wherein at least one of the struts of the second subset is not structurally integral with any of the struts of the first subset.
Inventive concept 80. The apparatus according to any one of Inventive concepts 59-67,

wherein the fluid flow guide is fixed to the first subset of the struts, the second subset of the struts, and a third subset of the struts so as to define the lumen, wherein the first, the second, and the third subsets do not include any common struts, and

wherein the struts of the third subset are elastic such that, when the stent-graft assumes the radially-expanded state, the struts of the third subset cause the fluid flow guide to define a flared axial portion that extends to one end of the fluid flow guide, the flared axial portion having (a) a greatest flared radius from the central longitudinal axis, which greatest flared radius is at least 5% greater than an average radius of the substantially cylindrical tubular portion axially adjacent the flared axial portion, and (b) an axial length equal to between 5% and 20% of a difference between (i) the greatest flared radius and (ii) the radius of the substantially cylindrical tubular portion axially adjacent the flared axial portion.

Inventive concept 81. The apparatus according to Inventive concept 80,

wherein the flared axial portion is a first flared axial portion, and the one end of the fluid flow guide is a first end of the fluid flow guide, and

wherein the struts of the third subset are elastic such that, when the stent-graft assumes the radially-expanded state, the struts of the third subset cause the fluid flow guide to additionally define a second flared axial portion that extends to a second end of the fluid flow guide, the second flared axial portion having (a) a greatest flared radius from the central longitudinal axis, which greatest flared radius is at least 5% greater than an average radius of the substantially cylindrical tubular portion axially adjacent the second flared axial portion, and (b) an axial length equal to between 5% and 20% of a difference between (i) the greatest flared radius and (ii) the radius of the substantially cylindrical tubular portion axially adjacent the second flared axial portion.

Inventive concept 82. The apparatus according to any one of Inventive concepts 59-67,

wherein the stent-graft further includes sutures that secure the struts of the first and the second subsets to the fluid flow guide, and

wherein at least 80% of a surface area of the struts of the first subset is within 3 mm of at least one of the sutures that secure the struts of the first subset to the fluid flow guide.

Inventive concept 83. The apparatus according to Inventive concept 82, wherein no more than 50% of the surface area of the struts of the second subset is within 3 mm of at least one of the sutures that secure the struts of the second subset to the fluid flow guide.
Inventive concept 84. The apparatus according to any one of Inventive concepts 59-67, wherein respective circumferences of all substantially cylindrical tubular portions of the fluid flow guide vary by less than 10%.
Inventive concept 85. The apparatus according to any one of Inventive concepts 59-67, wherein a circumference of a first one of the substantially cylindrical tubular portions is at least 10% greater than a circumference of a second one of the substantially cylindrical tubular portions.
Inventive concept 86. The apparatus according to any one of Inventive concepts 59-67, wherein an axial length of the bulge equals between 10% and 40% of a difference between (a) the greatest bulge radius and (b) the average radius of the substantially cylindrical tubular portions proximally and distally adjacent the bulge.
Inventive concept 87. The apparatus according to any one of Inventive concepts 59-67, wherein when the endovascular stent-graft is removably disposed in the delivery catheter in the radially-compressed state, the struts of the first subset do not coincide with the struts of the second subset.
Inventive concept 88. The apparatus according to any one of Inventive concepts 59-67,

wherein the stent-graft is a main stent-graft,

wherein the fluid flow guide is not shaped so as to define any fenestrations or scallops, and

wherein the apparatus further includes one or more branching stent-grafts.

Inventive concept 89. The apparatus according to any one of Inventive concepts 59-67,

wherein the stent-graft is a main stent-graft,

wherein the fluid flow guide is not shaped so as to define one or more openings selected from the group of openings consisting of: fenestrations, scallops, and fenestrations and scallops, and

wherein the apparatus further includes a number of branching stent-grafts, the number greater than a number of the openings.

There is additionally provided, in accordance with an Inventive concept 90 of the present invention, apparatus for use with a delivery catheter, the apparatus including an endovascular stent-graft, which is configured to initially be positioned in the delivery catheter in a radially-compressed state, and to assume a radially-expanded state upon being deployed from the delivery catheter, and which includes:

struts; and

a fluid flow guide, which includes at least one biologically-compatible substantially blood-impervious fabric, and which is fixed to first, second, and third subsets of the struts, wherein the first, the second, and the third subsets do not include any common struts,

wherein the struts of the first, the second, and the third subsets are elastic such that, when the stent-graft assumes the radially-expanded state:
the fluid flow guide defines a lumen having a central longitudinal axis,
the struts of the first subset cause the fluid flow guide to define a plurality of substantially cylindrical tubular portions,
the struts of the second subset cause the fluid flow guide to define a bulge having a greatest bulge radius from the central longitudinal axis, which greatest bulge radius is at least 5% greater than an average radius of the substantially cylindrical tubular portions proximally and distally adjacent the bulge, and
the struts of the third subset cause the fluid flow guide to define a flared axial portion that extends to one end of the fluid flow guide, the flared axial portion having (a) a greatest flared radius from the central longitudinal axis, which greatest flared radius is at least 5% greater than a radius of the substantially cylindrical tubular portion axially adjacent the flared axial portion, and (b) an axial length equal to between 5% and 20% of a difference between (i) the greatest flared radius and (ii) the radius of the substantially cylindrical tubular portion axially adjacent the flared axial portion.
   Inventive concept 91. The apparatus according to Inventive concept 90,

wherein the flared axial portion is a first flared axial portion, and the one end of the fluid flow guide is a first end of the fluid flow guide, and

wherein the struts of the third subset are elastic such that, when the stent-graft assumes the radially-expanded state, the struts of the third subset cause the fluid flow guide to additionally define a second flared axial portion that extends to a second end of the fluid flow guide, the second flared axial portion having (a) a greatest flared radius from the central longitudinal axis, which greatest flared radius is at least 5% greater than a radius of the substantially cylindrical tubular portion axially adjacent the second flared axial portion, and (b) an axial length equal to between 5% and 20% of a difference between (i) the greatest flared radius and (ii) the radius of the substantially cylindrical tubular portion axially adjacent the second flared axial portion.

Inventive concept 92. The apparatus according to Inventive concept 90,

wherein the bulge is a first bulge,

wherein the fluid flow guide is fixed to the first subset of the struts, the second subset of the struts, and a third subset of the struts so as to define the lumen, wherein the first, the second, and the third subsets do not include any common struts, and

wherein the struts of the third subset are elastic such that, when the stent-graft assumes the radially-expanded state, the struts of the third subset cause the fluid flow guide to define a second bulge having a greatest bulge radius from the central longitudinal axis, which greatest bulge radius is at least 5% greater than an average radius of the substantially cylindrical tubular portions proximally and distally adjacent the second bulge.

Inventive concept 93. The apparatus according to Inventive concept 90, wherein an axial length of the bulge equals between 10% and 40% of a difference between (a) the greatest bulge radius and (b) the average radius of the substantially cylindrical tubular portions proximally and distally adjacent the bulge.
Inventive concept 94. The apparatus according to Inventive concept 90, wherein a circumference of a first one of the substantially cylindrical tubular portions is at least 10% greater than a circumference of a second one of the substantially cylindrical tubular portions.
Inventive concept 95. The apparatus according to Inventive concept 90, wherein the bulge completely circumferentially encircles the stent-graft.
Inventive concept 96. The apparatus according to Inventive concept 90, wherein the struts of the first and the second subsets are superelastic.
Inventive concept 97. The apparatus according to any one of Inventive concepts 90-96,

wherein the stent-graft is a main stent-graft,

wherein the fluid flow guide is not shaped so as to define any fenestrations or scallops, and

wherein the apparatus further includes one or more branching stent-grafts.

Inventive concept 98. The apparatus according to any one of Inventive concepts 90-96,

wherein the stent-graft is a main stent-graft,

wherein the fluid flow guide is not shaped so as to define one or more openings selected from the group of openings consisting of: fenestrations, scallops, and fenestrations and scallops, and

wherein the apparatus further includes a number of branching stent-grafts, the number greater than a number of the openings.

There is yet additionally provided, in accordance with an Inventive concept 99 of the present invention, a method including:

advancing, into a main artery of a subject, an endovascular stent-graft, which is removably disposed in a delivery catheter in a radially-compressed delivery state, and includes (a) a main stent-graft, which includes (i) elastic struts and (ii) a fluid flow guide, which includes at least one biologically-compatible substantially blood-impervious fabric, and which is fixed to first and second subsets of the struts, wherein the first and the second subsets do not include any common struts;

deploying the endovascular stent-graft from the delivery catheter such that the endovascular stent-graft assumes a radially-expanded state in which (a) the fluid flow guide defines a lumen having a central longitudinal axis, (b) the struts of the first subset are arranged as a plurality of circumferential cells in circumferentially-continuous rings, which cause the fluid flow guide to define a plurality of substantially cylindrical tubular portions, (c) the struts of the second subset cause the fluid flow guide to define a bulge having a greatest bulge radius from the central longitudinal axis, which greatest bulge radius is at least 5% greater than an average radius of the substantially cylindrical tubular portions proximally and distally adjacent the bulge, (d) the struts of the second subset define tip portions, and (e) the number of the tip portions of the struts of the second subset that define the bulge is at least 50% greater than the average number of circumferential cells in the two circumferentially-continuous rings proximally and distally adjacent the bulge; and

deploying one or more branching stent-grafts partially alongside the main stent-graft and partially in respective branching arteries that branch from the main artery, such that portions of the branching stent-grafts contact the bulge.

There is also provided, in accordance with an Inventive concept 100 of the present invention, apparatus for use with a delivery catheter, the apparatus including an endovascular stent-graft, which is configured to initially be positioned in the delivery catheter in a radially-compressed state, and to assume a radially-expanded state upon being deployed from the delivery catheter, and which includes:

struts;

a fluid flow guide, which includes at least one biologically-compatible substantially blood-impervious fabric, and which is fixed to the struts; and

one or more bulges, which bulge radially outward and are arranged as one or more respective circumferential helices, wherein each of the circumferential helices circumscribes at least 0.3 complete turns around the endovascular stent-graft.

Inventive concept 101. The apparatus according to Inventive concept 100, wherein each of the one or more circumferential helices circumscribes at least 0.5 complete turns around the endovascular stent-graft.
Inventive concept 102. The apparatus according to Inventive concept 101, wherein each of the one or more circumferential helices circumscribes at least 1.5 complete turns around the endovascular stent-graft.
Inventive concept 103. The apparatus according to Inventive concept 100, wherein the one or more bulges is exactly one bulge.
Inventive concept 104. The apparatus according to Inventive concept 100, wherein the one or more bulges is a plurality of bulges, arranged as a plurality of helices.
Inventive concept 105. The apparatus according to Inventive concept 104, wherein the plurality of helices are arranged as an n-tuple helix.
Inventive concept 106. The apparatus according to any one of Inventive concepts 100-105, wherein each of the one or more bulges has a greatest bulge radius from a central longitudinal axis of the fluid flow guide, which greatest bulge radius is at least 5% greater than an average radius of substantially cylindrical tubular portions of the endovascular stent-graft.
Inventive concept 107. The apparatus according to any one of Inventive concepts 100-105, wherein the struts are shaped so as to define a lumen of the fluid flow guide, and wherein the one or more bulges are defined by the fabric of the fluid flow guide and the struts that are shaped so as to define the lumen of the fluid flow guide.
Inventive concept 108. The apparatus according to any one of Inventive concepts 100-105, wherein each of the one or more bulges includes at least one biologically-compatible substantially blood-impervious fabric, distinct from the fabric of the fluid flow guide.
Inventive concept 109. The apparatus according to Inventive concept 108, wherein each of the one or more bulges further includes one or more springs that provide structure to the one or more bulges.
Inventive concept 110. The apparatus according to Inventive concept 109, wherein the one or more springs include helical springs, and the fabric of the one or more bulges surrounds a portion of the springs, such that the springs are disposed between the fabric of the one or more bugles and the fluid flow guide.

The present invention will be more fully understood from the following detailed description of embodiments thereof, taken together with the drawings, in which:

### BRIEF DESCRIPTION OF THE DRAWINGS

FIGS. 1A-C are schematic illustrations of an endovascular stent-graft, in accordance with an application of the present invention;

FIGS. 2A-D are schematic illustration of exemplary deployments of an endovascular system in an aneurysmal descending aorta, in accordance with respective applications of the present invention;

FIG. 3 is a schematic illustration of cutting pattern for the fabric of a fluid flow guide of the endovascular stent-graft of FIGS. 1A-C during fabrication, in accordance with an application of the present invention;

FIG. 4 is a schematic illustration of a single undulating circumferentially-continuous ring of the endovascular stent-graft of FIGS. 1A-C, in accordance with an application of the present invention;

FIGS. 5A-B are schematic illustrations of alternative configurations of a single undulating circumferentially-continuous ring of the endovascular stent-graft of FIGS. 1A-C, in accordance with respective applications of the present invention;

FIG. 6 is a schematic illustration of another endovascular stent-graft, in accordance with an application of the present invention;

FIG. 7 is a schematic illustration of an exemplary deployment of an endovascular system in an aneurysmal descending aorta, in accordance with an application of the present invention;

FIG. 8 is a schematic illustration of yet another endovascular stent-graft, in accordance with an application of the present invention;

FIGS. 9A-B are schematic illustrations of still another endovascular stent-graft, in accordance with an application of the present invention;

FIGS. 10A-B are schematic illustrations of a single undulating circumferentially-continuous ring of an endovascular stent-graft when the stent-graft assumes a radially-compressed delivery state and a radially-expanded state, respectively, in accordance with an application of the present invention; and

FIGS. 11A-B are schematic illustrations of another endovascular stent-graft, in accordance with respective applications of the present invention.

### DETAILED DESCRIPTION OF APPLICATIONS

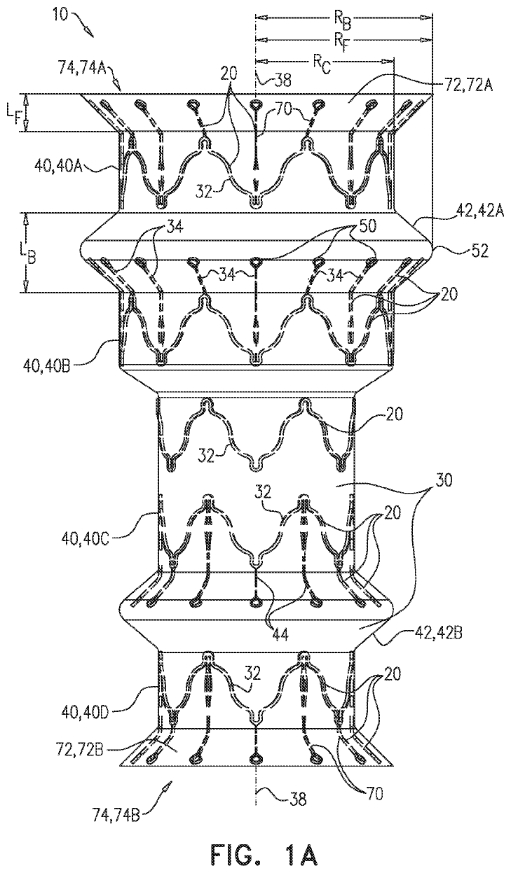

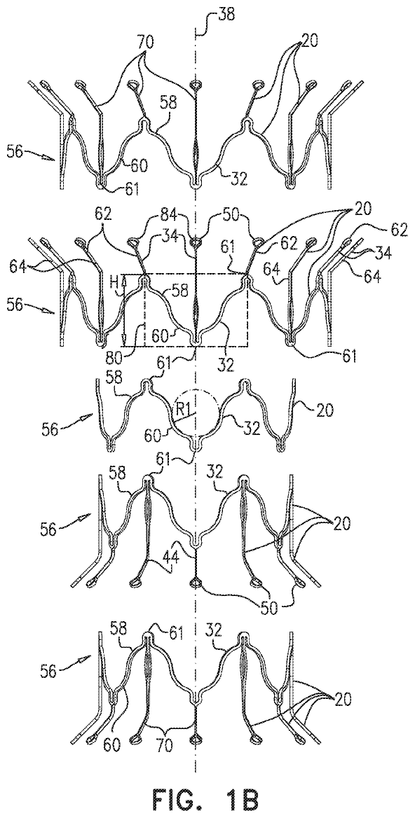

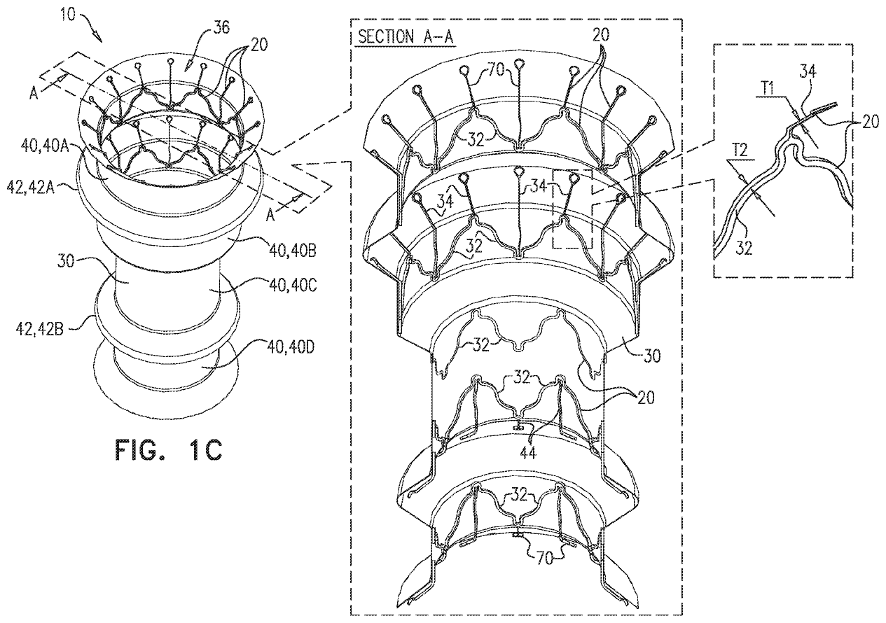

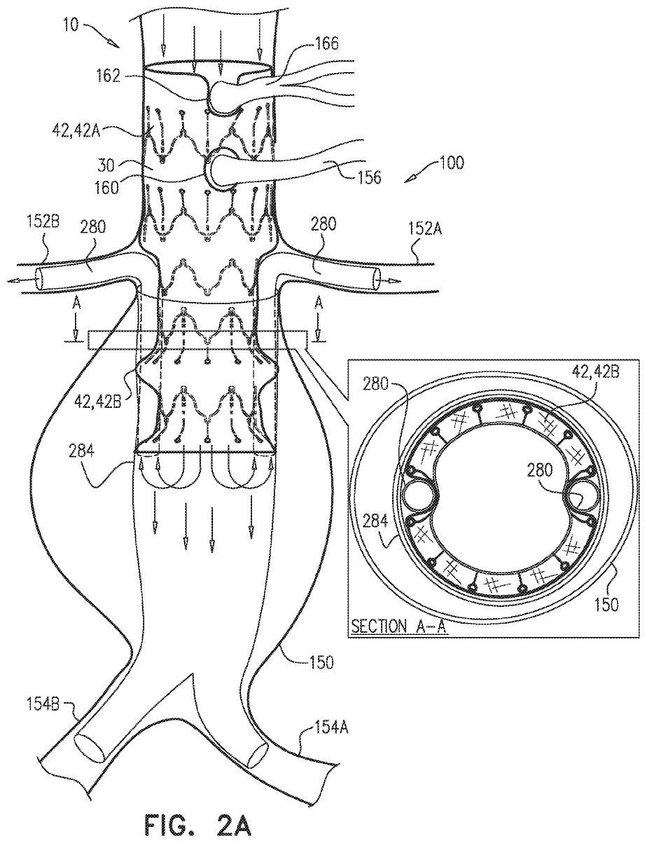

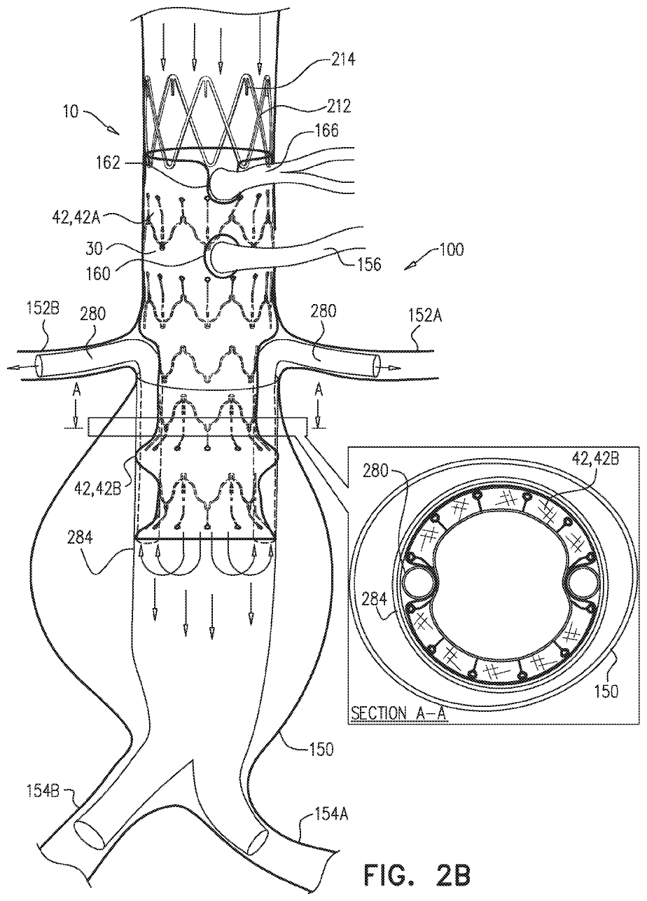

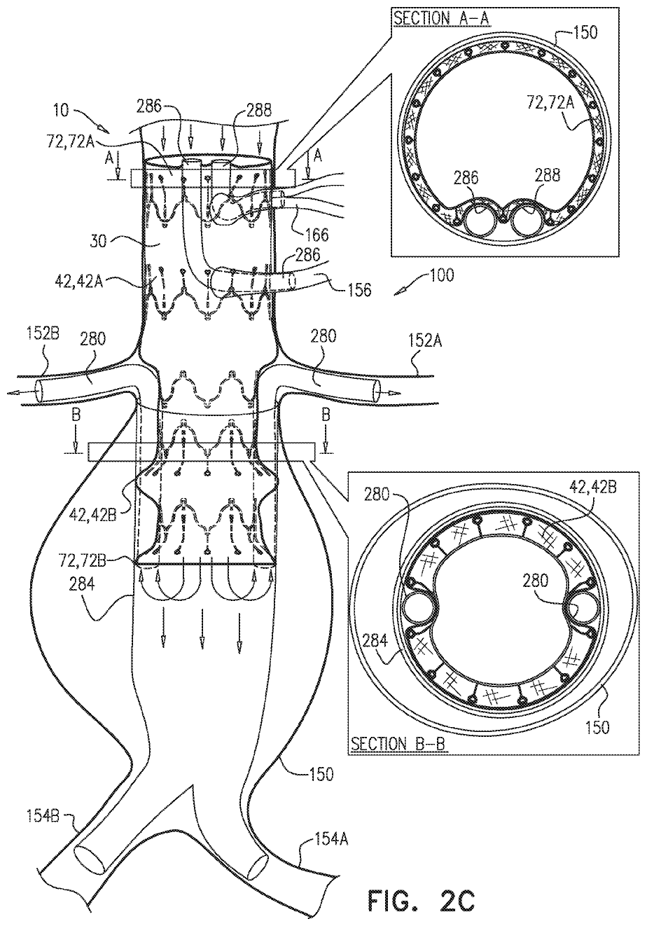

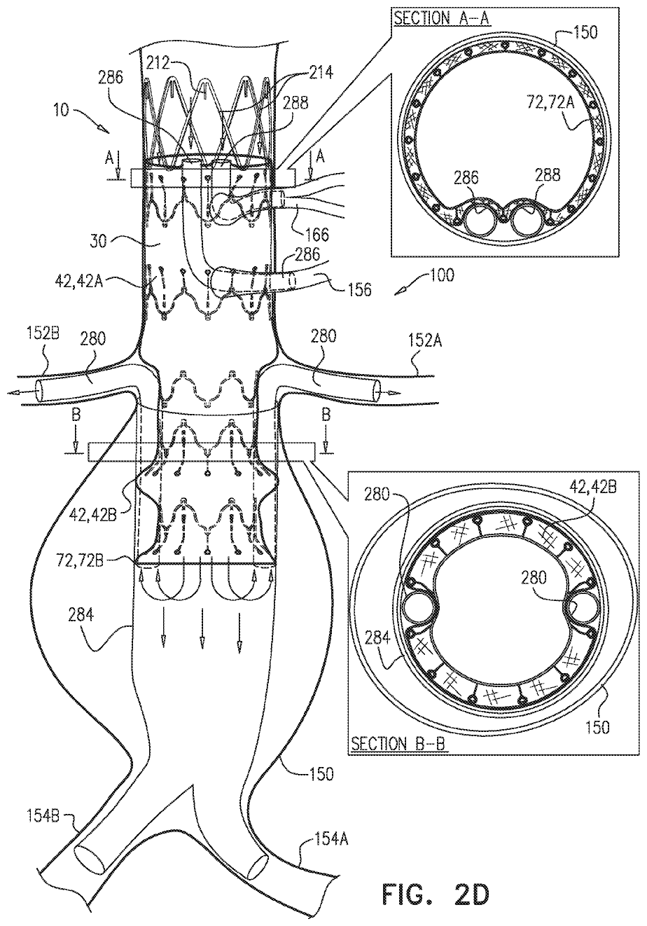

FIGS. 1A-C are schematic illustrations an endovascular stent-graft 10, in accordance with an application of the present invention. Endovascular stent-graft 10 may be provided as part of an endovascular system, which may additionally comprise other components, such as an endovascular delivery tool (e.g., comprising a delivery catheter), and/or additional endovascular stent-grafts, such as described hereinbelow with reference to FIGS. 2A-D. The endovascular system may be used to treat a blood vessel, such as an artery, e.g., descending aorta 150, suffering from an aneurysm, a dissection, or, more generally, a pathologically dilated blood vessel.

Endovascular stent-graft 10 is configured to initially be positioned in the delivery catheter in a radially-compressed state, and to assume a radially-expanded state upon being deployed from the delivery catheter, such as shown in FIGS. 1A-C and 2A-D. For some applications, endovascular stent-graft 10 is self-expanding, i.e., is configured to automatically transition from the radially-compressed delivery state to the radially-expanded state upon being released from the delivery catheter. For other applications, stent-graft 10 is plastically expandable, such as balloon-expandable.

Endovascular stent-graft 10 comprises:
struts 20 (i.e., elongate segments that provide structure to stent-graft 10, and are optionally interconnected with one another at respective junctions; optionally, a plurality of struts 20 are fabricated from a cylindrical tube, such as by laser cutting); and
a fluid flow guide 30, which comprises at least one biologically-compatible substantially blood-impervious fabric, and which is fixed to first and second subsets 32 and 34 of struts 20; first and second subsets 32 and 34 do not include any common struts 20.

Struts 20 of first and second subsets 32 and 34 are elastic such that, when stent-graft 10 assumes the radially-expanded state, such as shown in FIGS. 1A-C:
fluid flow guide 30 defines a lumen 36 (labeled in FIG. 1C) having a central longitudinal axis 38 (labeled in FIGS. 1A-B),
struts 20 of first subset 32 cause fluid flow guide 30 to define a plurality of substantially cylindrical tubular portions 40, and
struts 20 of second subset 34 cause fluid flow guide 30 to define a bulge 42 having a greatest bulge radius R_{B} from central longitudinal axis 38, which greatest bulge radius R_{B} is at least 5% greater (e.g., at least 10% greater, such as at least 15% greater) than an average radius R_{C} of substantially cylindrical tubular portions 40A and 40B proximally and distally adjacent bulge 42 (i.e., axially surrounding bulge 42).

For some applications, bulge 42 is configured to reduce the likelihood of long-term leakage (i.e., blood flow) through gutters, i.e., the residual intravascular space disposed outside the lumens of endovascular stent-graft 10 and branching stent-grafts disposed alongside endovascular stent-graft 10. Alternatively or additionally, bulge 42 may reduce the likelihood of blood flow between endovascular stent-graft 10 and features of the anatomy of the blood vessel wall, such as isolated regions of plaque, calcifications, or thrombus, all of which alter the circularity of the blood vessel wall and might otherwise present issues for good sealing between the stent-graft and the blood vessel wall. As a result, the likelihood of type 1 endoleak is reduced.

For some applications, an axial length L_{B} (labeled in FIG. 1A) of bulge 42 equals at least 10%, no more than 40%, and/or between 10% and 40% of a difference between (a) the greatest bulge radius R_{B} and (b) the average radius R_{C} of substantially cylindrical tubular portions 40A and 40B proximally and distally adjacent bulge 42 (i.e., axially surrounding bulge 42).

As used in the present application, including in the claims, the “central longitudinal axis” 38 of lumen 36 is the set of all centroids of transverse cross-sectional sections of lumen 36 along lumen 36. Thus the cross-sectional sections are locally perpendicular to central longitudinal axis 38, which runs along lumen 36. (For configurations in which lumen 36 is circular in cross-section, the centroids correspond with the centers of the circular cross-sectional sections.)

Typically, struts 20 comprise a metal, such as a flexible metal, an elastic metal, stainless steel (e.g., elastic stainless steel), cobalt-chromium, or a superelastic alloy (such as Nitinol). Fluid flow guide 30 comprises at least one biologically-compatible substantially blood-impervious fabric (e.g., one or more thin flexible sheets), which are typically arranged as a tubular structure (with a diameter that varies therealong), when stent-graft 10 is unconstrained in the radially-expanded state. The fabric may comprise, for example, a polymeric material (e.g., a polyester, or polytetrafluoroethylene (PT F E)), a textile material (e.g., polyethylene terephthalate (PET), e.g., Dacron÷, manufactured by E. I. du Pont de Nemours and Company, Wilmington, Del., USA), or expanded polytetrafluoroethylene (ePTFE), e.g., manufactured by W. L. Gore & Associates, Newark, Del., USA), natural tissue (e.g., saphenous vein or collagen), or a combination thereof.

Typically, fluid flow guide 30 is arranged such that all of the fabric defines lumen 36 when stent-graft 10 assumes the radially-expanded state (such that, for example, stent-graft 10 does not comprise any skirts that extend radially outward from fluid flow guide 30).

For some applications, struts 20 of first and second subsets 32 and 34 are arranged and shaped such that struts 20 of first subset 32 apply a radially-outward force that is at least 20% (e.g., at least 40%) greater than a radially-outward applied by struts 20 of second subset 34.

For some applications, as shown in FIGS. 1A-C, fluid flow guide 30 is shaped so as to define one or more additional bulges 42. For some applications, bulge 42 is a first bulge 42A, and fluid flow guide 30 is fixed to first subset 32 of struts 20, second subset 34 of struts 20, and a third subset 44 of struts 20 so as to define lumen 36; first subset 32, second subset 34, and third subset do not include any common struts 20. Struts 20 of third subset 44 are elastic such that, when stent-graft 10 assumes the radially-expanded state, struts 20 of third subset 44 cause fluid flow guide 30 to define a second bulge 42B having a greatest bulge radius from central longitudinal axis 38, which greatest bulge radius is at least 5% greater than an average radius of substantially cylindrical tubular portions 40C and 40D proximally and distally adjacent second bulge 42B (i.e., axially surrounding bulge 42B). First bulge 42A may prevent leakage from a chimney (i.e., from above), and second bulge 42B may prevent retrograde leakage from a “periscope” (i.e., from below), such as described hereinbelow with reference to FIGS. 2A-D.

For some applications, bulge 42 completely circumferentially encircles stent-graft 10. For other applications, bulge 42 only partially circumferentially encircles stent-graft 10. For some applications in which fluid flow guide 30 defines a plurality of bulges, along an entire axial length of fluid flow guide 30, each circumferential angle is at least circumscribed by one bulge 42. For some applications in which fluid flow guide 30 defines a plurality of bulges, along an entire axial length of fluid flow guide 30, each circumferential angle is at least circumscribed by two bulges 42.

For some applications, at least a first one of struts 20 of second subset 34 defines a first tip portion 50. For some applications, an angle is defined by (a) central longitudinal axis 38 and (b) a line defined by (i) the first tip portion 50 and (ii) a junction between the first one of struts 20 of second subset 34 and at least a second one of struts 20 of first subset 32. The angle is between 5 and 60 degrees, such as between 10 and 30 degrees. For some applications, the first tip portion 50 is disposed axially near (e.g., within 2 mm of) an axial location 52 along stent-graft 10 corresponding with the greatest bulge radius R_{B}, while for other applications, the first tip portion 50 axially crosses bulge 42 and terminates more than 2 mm from axial location 52. As used in the present application, including in the claims, a “tip portion” is a free end of a strut 20 of second subset 34; the free end is disposed radially outward from the substantially cylindrical tubular portion 40 from which the tip portion extends, when stent-graft 10 assumes the radially-expanded state. By “free end” it is meant a free end from other portions of struts 20; the tip portion may be sutured to fluid flow guide 30, as described hereinbelow.

For some applications, the first one of struts 20 of second subset 34 has a length measured along the first one of struts 20 between (a) the first tip portion 50 and (b) a junction between the first one of struts 20 of second subset 34 and the second one of struts 20 of first subset 32. A far half of the first one of struts 20 extends from the first tip portion 50 along 50% of the length of the first one of struts 20 of second subset 34. Stent-graft 10 further comprises sutures that secure struts 20 of first subset 32 and second subset 34 to fluid flow guide 30. None of the sutures are disposed along at least 50% of the far half of the first one of struts 20 of second subset 34. This configuration provides a degree of freedom of motion for the struts of the second subset. This freedom allows the struts of the second subset to better accommodate the disturbance of a branching stent-graft, which may provide better sealing between the bulge and the branching stent-graft. In this configuration, atraumatic features 84, described hereinbelow, typically do not serve as suture rings.

For some applications, as labeled in FIG. 1B, struts 20 of first subset 32 are arranged in a plurality of undulating circumferentially-continuous rings 56 having alternating peaks 58 and troughs 60 (i.e., apices pointing in opposite axial directions). For some applications, peaks 58 and troughs 60 are substantially semicircular, have a first average radius of curvature R1, and are shaped so as to define respective strain-distribution features 61 having a second average radius of curvature equal to between 10% and 50% of the first average radius of curvature. Optionally, strain-distribution features 61 are disposed on proximal-most and/or distal-most points of peaks 58 and/or troughs 60.

For some applications, a first plurality 62 of struts 20 of second subset 34 originate in (i.e., are joined to and extend from) a proximal half of one of undulating circumferentially-continuous rings 56, and a second plurality 64 of struts 20 of second subset 34 originate in a distal half of the one of the undulating circumferentially-continuous rings 56. Typically, struts 20 of the first plurality 62 and the second plurality 64 extend in the same axial direction (e.g., proximally, as shown in the set of struts 20 labeled in FIG. 1B) (although the struts also extend with a radially-outward directional component). For some applications, first plurality 62 of struts 20 of second subset 34 originate in a proximal 20% of an axial height H of the one of the undulating circumferentially-continuous rings 56, such as at proximal-most sites of the one of the undulating circumferentially-continuous rings 56. Alternatively or additionally, for some applications, second plurality 64 of struts 20 of second subset 34 originate in a distal 20% of the axial height H of the one of the undulating circumferentially-continuous rings 56, such as at distal-most sites of the one of the undulating circumferentially-continuous rings 56. For some applications, first plurality 62 of struts 20 of second subset 34 originate at two or more axially different locations of one of the undulating circumferentially-continuous rings 56.

Alternatively, for some applications, at least one of struts 20 of second subset 34 is not structurally integral with any of struts 20 of first subset 32 (configuration not shown).

For some applications, as labeled in FIG. 1A, fluid flow guide 30 is fixed to first subset 32 of struts 20, second subset 34 of struts 20, and a fourth subset 70 of struts 20 so as to define lumen 36; first, second, and fourth subsets 32, 34, and 70 do not include any common struts 20. Struts 20 of fourth subset 70 are elastic such that, when stent-graft 10 assumes the radially-expanded state, struts 20 of fourth subset 70 cause fluid flow guide 30 to define a flared axial portion 72 that extends to one end 74 of fluid flow guide 30, flared axial portion 72 having a greatest flared radius R_{F} from central longitudinal axis 38, which greatest flared radius R_{F} is at least 5% greater than a radius R_{C} of the substantially cylindrical tubular portion 40 axially adjacent flared axial portion 72. Typically, flared axial portion 72 has an axial length L_{F} equal to between 5% and 20% of a difference between (i) the greatest flared radius R_{F} and (ii) the radius R_{C} of the substantially cylindrical tubular portion 40 axially adjacent flared axial portion 72.

For some applications, flared axial portion 72 is a first flared axial portion 72A, and the one end 74 of fluid flow guide 30 is a first end 74A of fluid flow guide 30. Struts 20 of fourth subset 70 are elastic such that, when stent-graft 10 assumes the radially-expanded state, struts 20 of fourth subset 70 cause fluid flow guide 30 to additionally define a second flared axial portion 72B that extends to a second end 74B of fluid flow guide 30, second flared axial portion 72B having a greatest flared radius from central longitudinal axis 38, which greatest flared radius is at least 5% greater than a radius of the substantially cylindrical tubular portion 40 axially adjacent second flared axial portion 72B. Typically, second flared axial portion 72B has an axial length equal to between 5% and 20% of a difference between (i) the greatest flared radius and (ii) the radius of the substantially cylindrical tubular portion 40 axially adjacent second flared axial portion 72B.

Flared axial portions 72 may serve the same function as bulges 42. Providing flared axial portions 72 at the ends of stent-graft 10, rather than bulges 42 at the ends may serve to prevent blood from pooling and circulating in the bulges, which might cause thrombosis.

For some applications, stent-graft 10 further comprises sutures that secure struts 20 of first and second subsets 32 and 34 to fluid flow guide 30, and at least 80% of the surface area of struts 20 of first subset 32 is within 3 mm of at least one of the sutures that secure struts 20 of first subset 32 to fluid flow guide 30. For some applications, no more than 50% of the surface area of struts 20 of second subset 34 is within 3 mm of at least one of the sutures that secure struts 20 of second subset 34 to fluid flow guide 30.

For some applications, such as shown in FIG. 1C, struts 20 of second subset 34 have an average wall thickness T1, measured radially, that is no more than 80% (e.g., no more than 60%) of an average wall thickness T2 of struts 20 of first subset 32, measured radially. As used in the present application, including in the claims, the wall thickness of a strut is “measured radially” by measuring the distance between the surfaces of the strut that are farther and closer to central longitudinal axis 38 when the strut is oriented parallel with central longitudinal axis 38 (even though this is not necessarily its resting, unconstrained orientation). Such a lower wall thickness may contribute to a lower spring constant of struts 20 of second subset 34 than struts 20 of first subset 32, which may facilitate more local deformation (indentation) of bulge 42 around a branching stent-graft, without unnecessarily crushing the branching stent-graft, or necessitating internally reinforcement of the branching stent-graft with additional metallic stents, such as described hereinabove with reference to FIGS. 2A-D. For some applications, the lesser wall thickness T1 is achieved by electropolishing struts 20 of second subset 34 for a longer duration than struts 20 of first subset 32, thereby removing more material from those of the second subset. Alternatively or additionally, for some applications, (a) struts 20 of first subset 32 have a first average cross-sectional area, measured perpendicular to respective axes of struts 20 of first subset 32, (b) struts 20 of second subset 34 have a second average cross-sectional area, measured perpendicular to respective axes of struts 20 of second subset 34, and (c) the second average cross-sectional area is no more than 80% of the first cross-sectional area.

Typically, for applications in which stent-graft 10 defines second bulge 42B, such as described hereinabove, struts 20 of third subset 44 have an average wall thickness, measured radially, that is no more than 80% of the average wall thickness of struts 20 of first subset 32, measured radially.

Reference is still made to FIGS. 1A-C. For some applications, struts 20 of first subset 32 are arranged as a plurality of circumferential cells 80 in circumferentially-continuous rings 56, which cause fluid flow guide 30 to define the plurality of substantially cylindrical tubular portions 40 (for clarity of illustration, a single one of circumferential cells 80 is labeled in FIG. 1B). As shown in FIG. 1B, each of circumferential cells 80 circumferentially begins and ends at a same axial location, i.e., includes exactly one period (cycle) of struts 20 of first subset 32. It is noted that a period (cycle) is to be understood as the smallest possible repeating unit of struts 20, and thus two identical circumferentially adjacent cells define two periods, each having a certain width, rather than a single period having twice the certain width. (Optionally, not all of circumferentially-continuous rings 56 are connected to or associated with struts 20 that define a bulge 42 or a flared axial portion 72.) For some applications, as mentioned above, struts 20 of second subset 34 define tip portions 50 (which are optionally, but not necessarily, disposed axially near (e.g., within 2 mm of) axial location 52 along stent-graft 10 corresponding with the greatest bulge radius R_{B}). For some of these applications, the number of tip portions 50 of struts 20 of second subset 34 that define bulge 42 is at least 30% (typically at least 50%) greater than the average number of circumferential cells 80 in the two circumferentially-continuous rings 56 proximally and distally adjacent bulge 42 (i.e., axially surrounding bulge 42), such as least 175% greater, no more than 250% greater, and/or between 175% and 250% (e.g., 200%) of the average number of circumferential cells 80. For some applications, the number of the tip portions of struts 20 of second subset 34 that define bulge 42 equals at least 12, no more than 24, and/or between 12 and 24. Providing such a relative or absolute large number of tip portions 50 may facilitate more local deformation (indentation) of bulge 42 around a branching stent-graft, without unnecessarily crushing the branching stent-graft, or necessitating internally reinforcement of the branching stent-graft with additional metallic stents, such as described hereinbelow with reference to FIGS. 2A-D.

For some applications, each of circumferentially-continuous rings 56 is shaped so as to define between 4 and 16 circumferential cells 80. For some applications, at least 75% of circumferential cells 80 of each circumferentially-continuous ring 56 are similarly shaped. For some applications, the dimensions of circumferential cells 80 of each circumferentially-continuous ring 56 differ between circumferentially adjacent cells 80.

For some applications, tip portions 50 are directly sutured to fluid flow guide 30. For some of these applications, tip portions 50 are shaped so as to define respective atraumatic features 84 (some of which are labeled in FIG. 1B), which optionally, in some configurations, serve as suture rings which are directly sutured to fluid flow guide 30. Optionally, atraumatic features 84 are substantially circular, as shown. For some applications, at least some of tip portions 50 comprise respective radiopaque markers.

For some applications, circumferential cells 80 are W-shaped, V-shaped, U-shaped, or M-shaped (configurations not shown) or diamond-shaped (having a constant or varying width (configuration not shown).

Reference is now made to FIGS. 2A-D, which are schematic illustration of exemplary deployments of an endovascular system 100 in an aneurysmal descending aorta 150, in accordance with respective applications of the present invention. This “periscope” configuration in a “sandwich” assembly is typically used for treating dilations that are distal (i.e., downstream) to branching arteries, such as left and right renal arteries 152A and 152B. In this deployment, stent-graft 10 is a main stent-graft 10. Techniques for deployment may be used that are described in one or more of the patent applications incorporated hereinbelow by reference, or otherwise known in the art. Although the deployment is illustrated with reference to the descending aorta, renal arteries, SMA, and the celiac artery, endovascular system 100 may also be deployed in the vicinity of other main and branching blood vessels, such as arteries, e.g., visceral arteries.

In the configurations shown in FIGS. 2B and 2D, stent-graft 10 comprises a plurality of bare struts 212, such as described hereinabove with reference to FIG. 8.

FIGS. 2A-D show endovascular system 100 upon deployment of:
endovascular stent-graft 10 in descending aorta 150, spanning left and right renal arteries 152A and 152B,
two branching stent-grafts 280, positioned extending (a) side-by-side (i.e., in parallel with) a portion of endovascular stent-graft 10 distal (i.e., downstream) to branching left renal and right renal arteries 152A and 152B, and (b) into the renal arteries; these branching prostheses thus provide a blood-flow path from the main artery to the branching arteries. Second bulge 42B reduces the likelihood of long-term leakage (i.e., blood flow) between “gutters” between endovascular stent-graft 10 and branching stent-grafts 280; as a result, the likelihood of type 1 endoleak is reduced. In addition, the strut-structure of second bulge 42B may facilitate more local deformation (indentation) of the bulge around branching stent-graft 280, without unnecessarily crushing branching stent-grafts 280, or necessitating internally reinforcement of branching stent-grafts 280 with additional metallic stents, and
at least one extension endovascular stent-graft 284, which bypasses the aneurysmal sac to left and right iliac arteries 154A and 154B, and which is sealingly coupled to endovascular stent-graft 10 during the deployment procedure.

Typically, respective distal ends of branching stent-grafts 280 are disposed at or near a distal end of endovascular stent-graft 10, such as within 2 cm of the distal end of endovascular stent-graft 10 (either proximal or distal the distal end). Respective proximal ends of branching stent-grafts 280 are disposed in left and right renal arteries 152A and 152B.

In the configuration shown in FIGS. 2A and 2B, fluid flow guide 30 is shaped so as to define:
at least one fenestration 160 through fluid flow guide 30. For some applications, a perimeter of fenestration 160 equals between 20% and 80% (e.g., between 20% and 40%) of a perimeter of stent-graft 10 at an axial location of fenestration 160. For some applications, fenestration 160 is substantially circular, such as circular; and/or
at least one scallop 162. Typically, a circumference of scallop 162 is between 25 and 50 mm. For some applications, scallop 162 is U-shaped, V-shaped, rectangularly-shaped, or semicircularly shaped. For some applications, scallop 162 is disposed proximally (i.e., upstream) to fenestration 160.

In the configuration shown in FIGS. 2A and 2B, stent-graft 10 is disposed such that fenestration 160 provides a blood-flow path to superior mesenteric artery (SMA) 156 (which is on the anterior surface of the aorta), and scallop 162 provides a blood-flow path to celiac artery 166. (Thus, endovascular system 100 comprises a number of branching stent-grafts, the number greater than a number of the openings (fenestrations and/or scallops).

In the configuration shown in FIGS. 2C and 2D, fluid flow guide 30 is not shaped so as to define fenestration 160 or scallop 162. Instead, two additional branching stent-grafts 286 and 288 are deployed alongside a proximal portion of stent-graft 10 in a “chimney” configuration. First bulge 42A and/or first flared axial portion 72A prevent leakage from the chimney. In addition, the strut-structure of first bulge 42A may facilitate more local deformation (indentation) of the bulge around branching stent-grafts 286 and 288, without unnecessarily crushing branching stent-grafts 286 or 288, or necessitating internally reinforcement of branching stent-grafts 286 or 288 with additional metallic stents.

In the configurations shown in all of FIGS. 2A-D, upon deployment of all of the endoluminal stent-grafts, multi-component endovascular system 100 defines a blood-flow path from upstream of the renal arteries to the renal arteries, SMA, celiac artery, and iliac arteries. A combination of the configurations of FIGS. 2A, 2B, 2C, and 2D may be used.

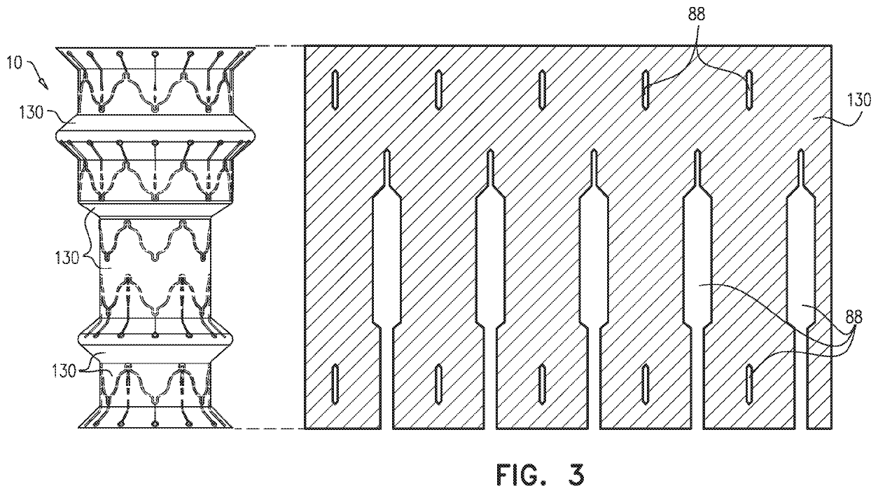

Reference is made to FIG. 3, which is a schematic illustration of cutting pattern for the fabric of fluid flow guide 30 during fabrication, in accordance with an application of the present invention. During fabrication of fluid flow guide 30, portions 88 of the fabric of fluid flow guide 30 are cut away, in order to provide fluid flow guide 30 with different diameters therealong corresponding to the differing diameters of stent-graft 10.

Reference is made to FIG. 4, which is a schematic illustration of a single undulating circumferentially-continuous ring 56, in accordance with an application of the present invention. In this view, undulating circumferentially-continuous ring 56 is shown cut at a circumferential site of one of struts 20 of first subset 32 and laid flat.

For some applications, when endovascular stent-graft 10 is removably disposed in the delivery catheter in the radially-compressed state, struts 20 of first subset 32 do not coincide with struts 20 of second subset 34. In other words, if struts 20 are cut at a circumferential site of one of struts 20 of first subset 32 and laid flat, as shown in FIG. 4, struts 20 of first subset 32 do not overlap struts 20 of second subset 34, i.e., do not occupy any of the same locations in the plane. This arrangement of struts 20 avoids the increased crossing profile in the radially-compressed delivery state that would result if the struts overlapped one another.

For some applications, struts 20 of first and second subsets 32 and 34 are fabricated from a single piece of a tubular material (e.g., by laser cutting the material). For some applications, at least one of struts 20 of second subset 34 is structurally integral with at least one of struts 20 of first subset 32, such as shown in the figures. For some applications, all of struts 20 of second subset 34 are structurally integral with at least one of struts 20 of first subset 32, none of struts 20 of second subset 34 is directly connected to any of the other struts of second subset 34, and none of struts 20 of second subset 34 is indirectly connected to any of the other struts of second subset 34 by any struts other than struts 20 of first subset 34. (Struts 20 of second subset 34 are typically indirectly connected by the fluid flow guide 30.) This arrangement of struts 20 allows each circumferential portion of bulge 42 to radially expand generally separately from one another, because circumferentially-adjacent end portions of struts 20 of second subset 34 do not pull on each other.

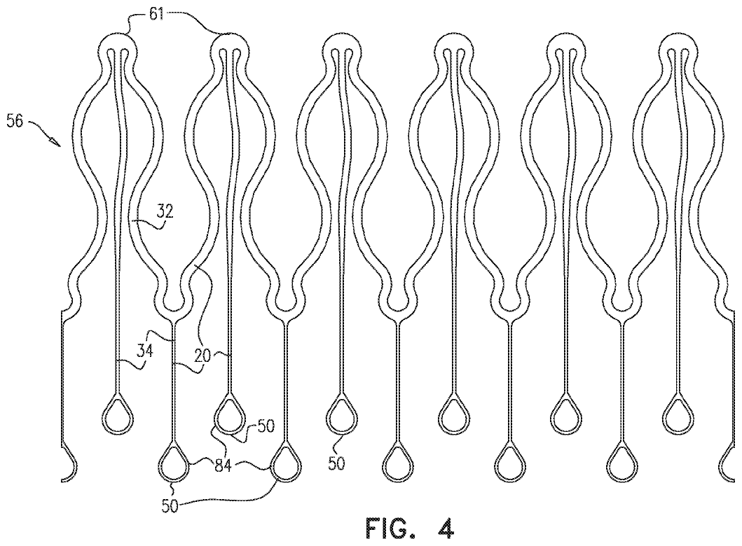

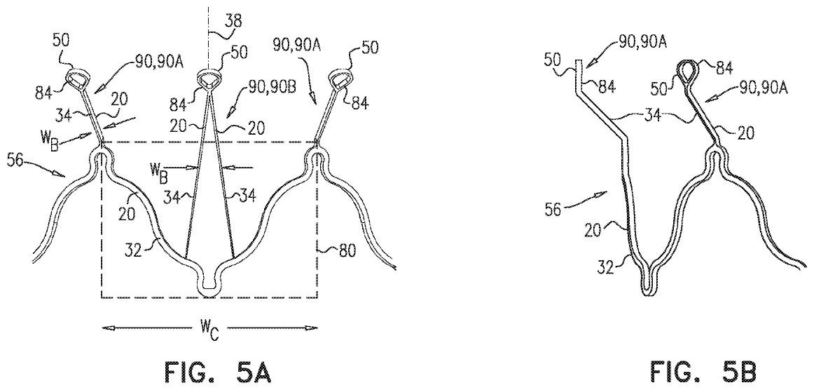

Reference is made to FIGS. 5A-B, which are schematic illustrations of alternative configurations of a single undulating circumferentially-continuous ring 56, in accordance with respective applications of the present invention. Only a portion of undulating circumferentially-continuous ring 56 is shown in FIGS. 5A-B. These configurations may be used with any of the configuration of stent-graft 10 described herein, mutatis mutandis.

In these configurations, struts 20 of second subset 34 are arranged as a plurality of bulge-inducing units 90 that cause fluid flow guide 30 to define bulge 42. Typically, an average circumferential width W_{B} of bulge-inducing units 90 is no more than 25% (e.g., no more than 20%, no more than 15%, or no more than 10%) of an average circumferential width W_{C} of circumferential cells 80 in the two circumferentially-continuous rings 56 proximally and distally adjacent bulge 42 (i.e., axially surrounding bulge 42) (only one of these two circumferentially-continuous rings 56 is shown in FIGS. 5A and 5B). The narrow widths of bulge-inducing units 90 may facilitate more local deformation (indentation) of bulge 42 around a branching-stent graft, without unnecessarily crushing the branching stent-graft, or necessitating internally reinforcement of the branching stent-graft with additional metallic stents, such as described hereinabove with reference to FIGS. 2A-D. (The “circumferential width” is measured around the circumference of undulating circumferentially-continuous ring 56, as though the ring were cut and laid flat.) For some applications, at least one of (e.g., all of) bulge-inducing units 90 has an aspect ratio (length/width) of at least 4.

For some applications, such as shown in FIG. 5A, all or a portion of bulge-inducing units 90 comprise two struts 20, as shown for bulge-inducing unit 90B, and/or all or a portion of bulge-inducing units 90 comprise exactly one strut 20, which is connected to first subset 32 at exactly one junction, as shown for bulge-inducing units 90A. (The average circumferential width W_{B} of bulge-inducing units 90B equals the weighted average circumferential width of tip portion 50 and the exactly one strut 20 of second subset 34.) FIG. 5A shows a configuration in which bulge-inducing units 90A alternate with bulge-inducing units 90A, while FIG. 5B shows a configuration with only bulge-inducing unit 90A. Thus, in some other configurations described herein (e.g., as shown in FIGS. 1A-C, 4, 6, 7, 8, and 9A-B), struts 20 of second subset 34 are arranged as a plurality of bulge-inducing units 90, each of which comprises exactly one strut 20 connected to first subset 32 at exactly one junction.

For some applications, the number of bulge-inducing units 90 is at least 30% (typically at least 50%) greater than the average number of circumferential cells 80 in the two circumferentially-continuous rings 56 proximally and distally adjacent bulge 42 (i.e., axially surrounding bulge 42), such as least 175% greater, no more than 250% greater, and/or between 175% and 250% (e.g., 200%) of the average number of circumferential cells 80.

For some applications, each of bulge-inducing units 90 circumscribes an arc having an angle of between 3 and 20 degrees, e.g., between 5 and 15 degrees.

As described hereinabove with reference to FIGS. 1A-C, for some applications, at least one of struts 20 of second subset 34 defines a tip portion 50. Tip portions 50 may be shaped so as to define respective atraumatic features 84, which are optionally substantially circular. For some applications, such as shown in FIG. 5B, atraumatic features 84 of tip portions 50 are oriented generally parallel to a cylindrical surface defined by first subset 32 of struts 20. This orientation may reduce the risk of damage to fluid flow guide 30 by tip portions 50 in bulges 42.

Reference is again made to FIGS. 1A-C. For some applications, a circumference of a first one of substantially cylindrical tubular portions 40 (e.g., 40A or 40B) is at least 10% greater than a circumference of a second one of substantially cylindrical tubular portions 40 (e.g., 40C or 40D).

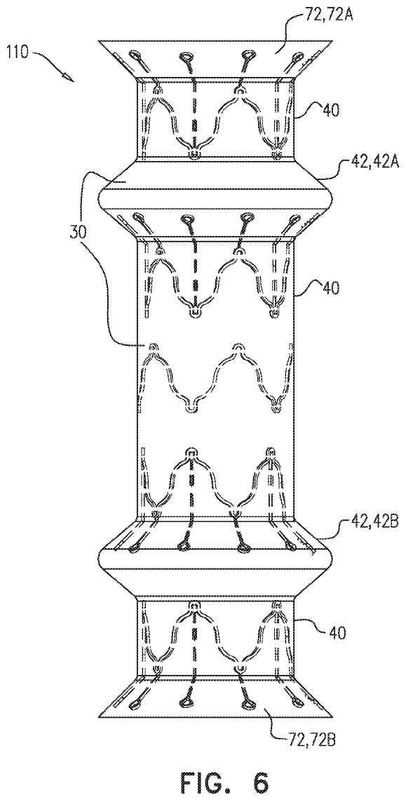

Reference is made to FIG. 6, which is a schematic illustration of an endovascular stent-graft 110, in accordance with an application of the present invention. Except as described below, endovascular stent-graft 110 is similar to endovascular stent-graft 10, described hereinabove with reference to FIGS. 1A-5B, and may implement any of the features thereof, mutatis mutandis. In endovascular stent-graft 110, respective circumferences of all substantially cylindrical tubular portions 40 of fluid flow guide 30 vary by less than 10%, such as are equal.

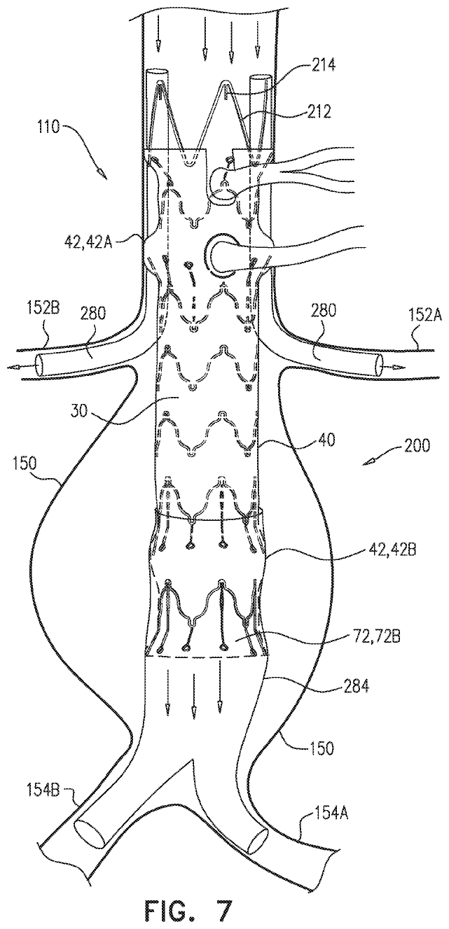

Reference is now made to FIG. 7, which is a schematic illustration of an exemplary deployment of an endovascular system 200 in aneurysmal descending aorta 150, in accordance with an application of the present invention. This “periscope” configuration in a “sandwich” assembly is typically used for treating dilations that are distal (i.e., downstream) to branching arteries, such as left and right renal arteries 152A and 152B. In this deployment, stent-graft 110 is a main stent-graft 110. Techniques for deployment may be used that are described in one or more of the patent applications incorporated hereinbelow by reference, or otherwise known in the art.

FIG. 7 shows endovascular system 200 upon deployment of:
endovascular stent-graft 110 in descending aorta 150, spanning left and right renal arteries 152A and 152B, after delivery catheter 772 has been withdrawn to release stent-graft 10, and
two branching stent-grafts 280, positioned partially extending along a proximal portion of endovascular stent-graft 110 in a “chimney” configuration and into respective branching arteries: left renal artery 152A and right renal artery 152B; first bulge 42A and/or first flared axial portion 72A reduce the likelihood of long-term leakage (i.e., blood flow) through “gutters.” As a result, the likelihood of type 1 endoleak is reduced.

Typically, respective proximal ends of branching stent-grafts 280 are disposed at or near a proximal end of endovascular stent-graft 110, such as within 2 cm of the proximal end of endovascular stent-graft 110 (either proximal or distal the proximal end). Preferably, the respective proximal ends of branching stent-grafts 280 are disposed not proximally to the proximal end of endovascular stent-graft 110, because if they were disposed proximally to the proximal end of endovascular stent-graft 110, blood flow might cause them to bend, curve, and whip in accordance with the aortic systole cycle. Respective distal ends of branching stent-grafts 280 are disposed in left and right renal arteries 152A and 1528.

Optionally, endovascular system 200 includes one or more extension endovascular stent-grafts 284, which collectively bypass the aneurysmal sac to left and right iliac arteries 154A and 1548. The extension endovascular prostheses are sealingly coupled to endovascular stent-graft 110 during the deployment procedure. For some applications, main stent-graft 110, other than at bulges 42, is oversized about 15% compared to extension endovascular stent-grafts 284. As can be seen in FIG. 7, upon deployment of all of the endoluminal prostheses, multi-component endovascular system 200 defines a blood-flow path from upstream of the renal arteries to the renal arteries, SMA, celiac artery, and iliac arteries. For some applications, a distal portion of extension endovascular stent-graft 284 is bifurcated, as shown in FIG. 7.

Reference is made to FIG. 8, which is a schematic illustration of an endovascular stent-graft 210, in accordance with an application of the present invention. Except as described below, endovascular stent-graft 210 is similar to endovascular stent-grafts 10 and 110, described hereinabove with reference to FIGS. 1A-5B and 6-7, respectively, and may implement any of the features thereof, mutatis mutandis. Endovascular stent-graft 210 comprises a plurality of bare struts 212 at a proximal end of the stent-graft. Optionally, one or more of bare struts 212 are shaped so as define fixation barbs 214, which may be oriented at an angle of at least 20 degrees with central longitudinal axis 38. For some applications, at least 10% (e.g., at least 50%) of an axial length of stent-graft 210 is not covered by fluid flow guide 30 (i.e., has only bare struts 212.

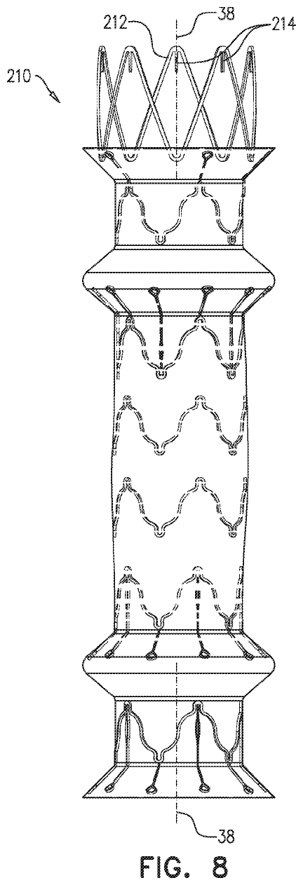

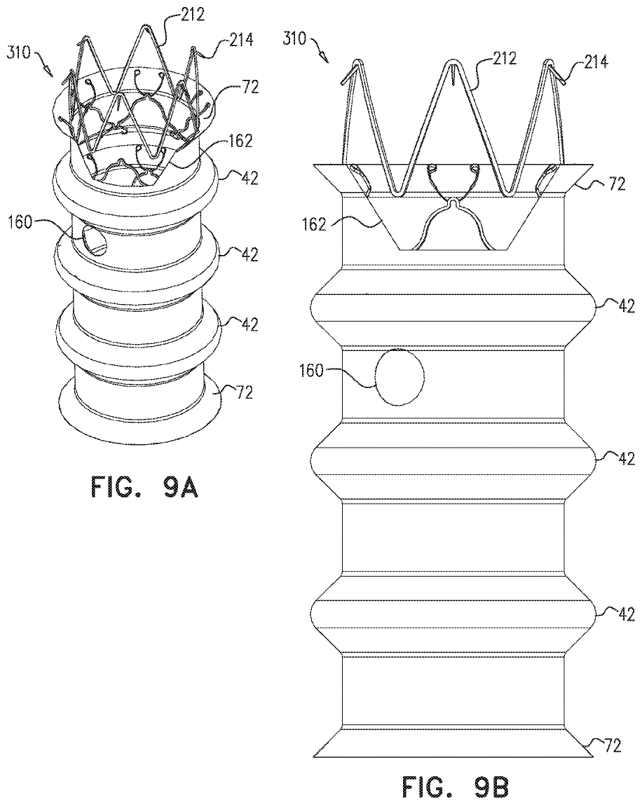

Reference is made to FIGS. 9A-B, which are schematic illustrations of an endovascular stent-graft 310, in accordance with an application of the present invention. Except as described below, endovascular stent-graft 310 is similar to endovascular stent-grafts 10, 110, and 210 described hereinabove with reference to FIGS. 1A-5B, 6-7, and 8, respectively, and may implement any of the features thereof, mutatis mutandis.

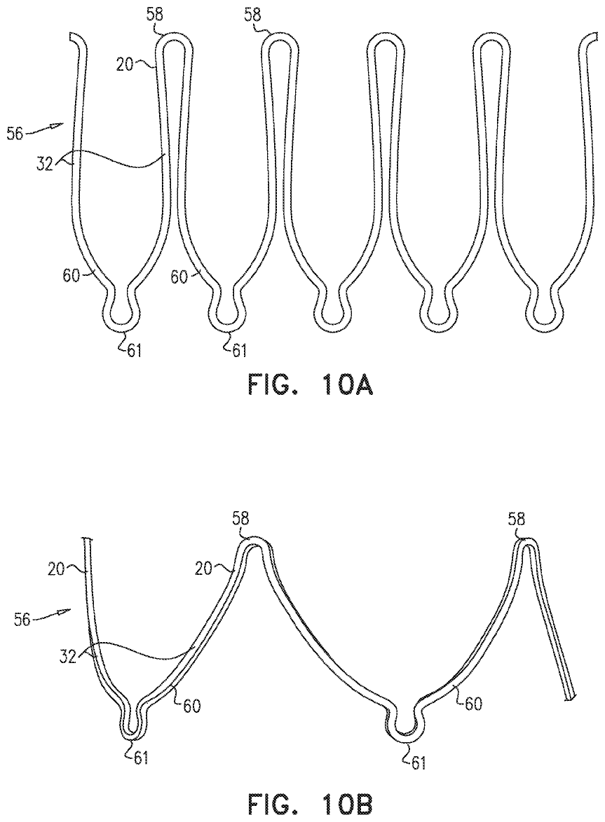

Reference is made to FIGS. 10A-B, which are schematic illustrations of a single undulating circumferentially-continuous ring 56 when stent-graft 10 assumes the radially-compressed delivery state and the radially-expanded state, respectively, in accordance with an application of the present invention. FIGS. 10A-B show only first subset 32 of struts 20, and do not show second subset 34 of struts 20, which are optionally provided, such as shown, for example, in FIGS. 1A-B, 4, 5A-B, and/or 11.

In the configuration shown in FIGS. 10A-B, unlike in the configuration shown, for example, in FIGS. 1A-B, only troughs 60 are shaped so as to define respective strain-distribution features 61. For some applications, peaks 58, and struts 20 of first subset 32 between peaks 58, together define a convex bell shape having a relatively large opening that can accommodate the aorta disturbance, by directly the disturbance into the opening. Alternatively, only peaks 58 are shaped so as to define respective strain-distribution features 61 (configuration not shown).

For some applications, as shown in FIGS. 1A-C, said array of elongated members is structurally integrated with struts 20 of first subset 32 via yet another substantially elongated member. For some applications, as shown in FIGS. 1A-C, an end of elongated member has a radius of curvature that is at least 150% of the lowest width of said member.

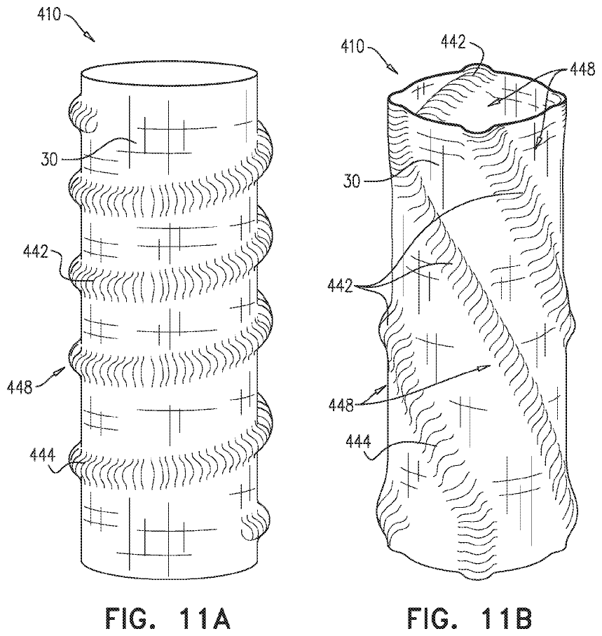

Reference is made to FIGS. 11A-B, which are schematic illustrations of an endovascular stent-graft 410, in accordance with respective applications of the present invention. Except as described below, endovascular stent-graft 410 is similar to endovascular stent-grafts 10, 110, 210, and 310 described hereinabove with reference to FIGS. 1A-5B, 6-7, 8, and 9A-B, respectively, and may implement any of the features thereof, mutatis mutandis. Endovascular stent-graft 410 comprises one or more bulges 442, which bulge radially outward (i.e., away from a central longitudinal axis of fluid flow guide 30), and are arranged as one or more respective circumferential helices 448, each of which circumscribes at least 0.3 complete turns, such as at least 0.5 complete turns, e.g., at least 1.5, 2, or 3 complete turns, around endovascular stent-graft 410.

For some applications, such as shown in FIG. 11A, endovascular stent-graft 410 comprises exactly one bulge 442.

For other applications, such as shown in FIG. 11B, endovascular stent-graft 410 comprises a plurality of bulges 442, e.g., exactly two bulges 442, exactly three bulges 442, exactly four bulges 442 (as shown in FIG. 11B), or five or more bulges 442. For example, the plurality of helices 448 may be arranged as a n-tuple helix, e.g., a double helix, a triple helix, or a quadruple helix (as shown in FIG. 11B). As used in the present application, including in the claims, an “n-tuple helix” comprises n helices with the same axis, differing by a translation along the axis.

For some applications, the one or more bulges 442 are configured to reduce the likelihood of long-term leakage (i.e., blood flow) through gutters, i.e., the residual intravascular space disposed outside the lumens of endovascular stent-graft 410 and branching stent-grafts disposed alongside endovascular stent-graft 410. Because the one or more bulges 442 are arranged as respective helices 448, the one or more bulges 442 contact the branching stent-grafts regardless of the circumferential location of the branching stent-grafts around endovascular stent-graft 410.

Alternatively or additionally, the one or more bulges 442 may reduce the likelihood of blood flow between endovascular stent-graft 410 and features of the anatomy of the blood vessel wall, such as isolated regions of plaque, calcifications, or thrombus, all of which alter the circularity of the blood vessel wall and might otherwise present issues for good sealing between stent-graft 410 and the blood vessel wall. As a result, the likelihood of type 1 endoleak is reduced.

For some application, such as shown in FIG. 11A, the pitch of the one or more helices 448 is less than an axial length of an axial portion of endovascular stent-graft 410 that comprises the one or more bulges 442, e.g., less than 50% of the axial length, e.g., less than 33% of the axial length. For other applications, such as shown in FIG. 11B, the pitch of the one or more helices 448 is greater than the axial length of the axial portion of endovascular stent-graft 410 that comprises the one or more bulges 442, e.g., greater than 150% of the axial length, e.g., greater than 200% of the axial length.

For some applications, such as shown in FIG. 11A, each of the one or more bulges 442 comprises at least one biologically-compatible substantially blood-impervious fabric 444. Fabric 444 is distinct from the fabric of fluid flow guide 30; for example, an inner surface of the fabric of fluid flow guide 30 defines a blood-flow path through fluid flow guide 30, while fabric 444 does not. The one or more bulges 442 are coupled to an external surface of fluid flow guide 30. Typically, each of the one or more bulges 442 further comprises one or more springs, which provide structure to the one or more bulges 442, and are disposed between fabric 444 of the one or more bulges and the fabric of fluid flow guide 30. For example, the one or more springs may comprise helical springs, and fabric 444 may surrounds a portion of the springs, such that the springs are disposed between fabric 444 and fluid flow guide 30. Typically, fabric 444 is sutured to fluid flow guide 30.

For other applications, such as shown in FIG. 11B, the one or more bulges 442 are defined by the fabric of fluid flow guide 30 and the same struts that define lumen 36 of fluid flow guide 30.

It is noted that the features of this configuration shown in FIG. 11B may be implemented in combination with the features shown in FIG. 11A, and vice versa.

Typically, each of the one or more bulges 442 has a greatest bulge radius from a central longitudinal axis of fluid flow guide 30 (labeled in FIG. 1C for endovascular stent-graft 10), which greatest bulge radius is at least 5% greater (e.g., at least 10% greater, such as at least 15% greater) than an average radius of substantially cylindrical tubular portions of endovascular stent-graft 410 (i.e., without including bulged portions of the endovascular stent-graft in the calculation of the average radius).

For some applications, the one or more bulges 442 extend to one or both ends of endovascular stent-graft 410, such as shown in FIG. 11B, while for other applications, the one or more bulges 442 do not extend to one or both ends of endovascular stent-graft 410, such as shown in FIG. 11A.

For some applications, the apparatus is substantially tubular on a first end portion and bifurcates to two substantially tubular sub-lumens on its second end portion, creating a continuous space between first end portion and sub-lumens on its second end portion. For some applications, an average circumference of each of its sub-lumens is between 40% and 70% of an average circumference of its first end portion.

Reference is again made to FIG. 3. For some applications, struts 20 of first subset 32 define a conical angle with respect to axis of said stent-graft, said conical angle between 10 to 30 degrees with respect to the angle of the lumen, when the apparatus is in its radially-expanded state.

In an embodiment, techniques and apparatus described in one or more of the following patents and patent applications, which are assigned to the assignee of the present application and are incorporated herein by reference, are combined with techniques and apparatus described herein.
PCT Application PCT/IL2008/000287, filed Mar. 5, 2008, which published as PCT Publication WO 2008/107885
PCT Application PCT/IB2010/052861, filed Jun. 23, 2010, which published as PCT Publication WO 2010/150208
PCT Application PCT/IL2010/000564, filed Jul. 14, 2010, which published as PCT Publication WO 2011/007354
PCT Application PCT/IL2010/000917, filed Nov. 4, 2010, which published as PCT Publication WO 2011/055364
PCT Application PCT/IL2010/000999, filed Nov. 30, 2010, which published as PCT Publication WO 2011/064782
PCT Application PCT/IL2010/001018, filed Dec. 2, 2010, which published as PCT Publication WO 2011/067764
PCT Application PCT/IL2010/001037, filed Dec. 8, 2010, which published as PCT Publication WO 2011/070576
PCT Application PCT/IL2011/000135, filed Feb. 8, 2011, which published as PCT Publication WO 2011/095979
PCT Application PCT/IL2012/000060, filed Feb. 2, 2012, which published as PCT Publication WO 2012/104842
PCT Application PCT/IL2012/000241, filed Jun. 19, 2012, which published as PCT Publication WO 2012/176187
PCT Application PCT/IL2012/000300, filed Aug. 12, 2012, which published as PCT Publication WO 2013/030819
U.S. Pat. No. 8,317,856 to Shalev et al.
U.S. Pat. No. 8,574,287 to Benary et al.
U.S. Provisional Application 60/892,885, filed Mar. 5, 2007
U.S. Provisional Application 60/991,726, filed Dec. 2, 2007
U.S. Provisional Application 61/219,758, filed Jun. 23, 2009
U.S. Provisional Application 61/221,074, filed Jun. 28, 2009
U.S. application Ser. No. 13/031,871, filed Feb. 22, 2011, which published as US Patent Application Publication 2011/0208289
U.S. Provisional Application 61/496,613, filed Jun. 14, 2011
U.S. Provisional Application 61/499,195, filed Jun. 21, 2011
U.S. Provisional Application 61/505,132, filed Jul. 7, 2011
U.S. Provisional Application 61/529,931, filed Sep. 1, 2011
U.S. Provisional Application 61/553,209, filed Oct. 30, 2011
U.S. Pat. No. 8,870,938 to Shalev et al.
U.S. application Ser. No. 13/384,075, filed Jan. 13, 2012, which published as US Patent Application Publication 2012/0179236
U.S. application Ser. No. 13/505,996, filed May 3, 2012, which published as US Patent Application Publication 2012/0310324
U.S. application Ser. No. 13/513,397, filed Jun. 1, 2012, which published as US Patent Application Publication 2012/0330399
U.S. application Ser. No. 13/514,240, filed Jun. 6, 2012, which published as US Patent Application Publication 2013/0013051
U.S. Provisional Application 61/678,182, filed Aug. 1, 2012
U.S. Pat. No. 9,468,517 to Shalev
U.S. Pat. No. 8,945,203 to Shalev et al.
U.S. application Ser. No. 13/807,880, filed Dec. 31, 2012, which published as US Patent Application Publication 2013/0131783
PCT Application PCT/IL2012/000095, filed Mar. 1, 2012, which published as PCT Publication WO 2012/117395
PCT Application PCT/IL2012/000148, filed Apr. 4, 2012, which published as PCT Publication WO 2013/030818
PCT Application PCT/IL2012/000190, filed May 15, 2012, which published as PCT Publication WO 2013/171730
PCT Application PCT/IL2012/000269, filed Jul. 2, 2012, which published as PCT Publication WO 2013/005207
PCT Application PCT/IL2012/050424, filed Oct. 29, 2012, which published as PCT Publication WO 2013/065040
PCT Application PCT/IL2012/050506, filed Dec. 4, 2012, which published as PCT Publication WO 2013/084235
U.S. Provisional Application 61/749,965, filed Jan. 8, 2013
U.S. Pat. No. 8,951,298 to Shalev
U.S. Provisional Application 61/775,964, filed Mar. 11, 2013
U.S. Provisional Application 61/826,544, filed May 23, 2013
U.S. Pat. No. 9,855,046 to Shalev
PCT Application PCT/IL2013/050656, filed Jul. 31, 2013, which published as PCT Publication WO 2014/020609
U.S. Provisional Application 61/906,014, filed Nov. 19, 2013
PCT Application PCT/IL2014/050019, filed Jan. 7, 2014, which published as PCT Publication WO 2014/108895
U.S. Provisional Application 61/926,533, filed Jan. 13, 2014
PCT Application PCT/IL2014/050174, filed Feb. 18, 2014, which published as PCT Publication WO 2014/141232
PCT Application PCT/IL2014/050434, filed May 18, 2014, which published as PCT Publication WO 2014/188412
PCT Application PCT/IL2014/050973, filed Nov. 6, 2014, which published as PCT Publication WO 2015/075708
U.S. Provisional Application 62/093,497, filed Dec. 18, 2014
U.S. Provisional Application 62/102,265, filed Jan. 12, 2015
U.S. application Ser. No. 14/416,236, filed Jan. 21, 2015, which published as US Patent Application Publication 2015/0202065
U.S. Provisional Application 62/110,659, filed Feb. 2, 2015
U.S. Provisional Application 62/254,432, filed Nov. 12, 2015
PCT Application PCT/IL2015/051221, filed Dec. 16, 2015, which published as PCT Publication WO 2016/098113
PCT Application PCT/IL2016/050014, filed Jan. 6, 2016, which published as PCT Publication WO 2016/113731
PCT Application PCT/IL2016/050049, filed Jan. 14, 2016, which published as PCT Publication WO 2016/125137
U.S. Provisional Application 62/371,983, filed Aug. 8, 2016
PCT Application PCT/IL2016/051207, filed Nov. 9, 2016, which published as PCT Publication WO 2017/081679

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with a delivery catheter, the apparatus comprising an endovascular stent-graft, which is configured to initially be positioned in the delivery catheter in a radially-compressed state, and to assume a radially-expanded state upon being deployed from the delivery catheter, and which comprises:
struts; and
a fluid flow guide, which comprises at least one biologically-compatible substantially blood-impervious fabric, and which is fixed to first and second subsets of the struts, wherein the first and the second subsets do not include any common struts,
wherein the struts of the first and the second subsets are elastic such that, when the stent-graft assumes the radially-expanded state:
the fluid flow guide defines a lumen having a central longitudinal axis,
the struts of the first subset are arranged as a plurality of circumferential cells in circumferentially-continuous rings, which cause the fluid flow guide to define a plurality of substantially cylindrical tubular portions,
the struts of the second subset cause the fluid flow guide to define a bulge having a greatest bulge radius from the central longitudinal axis, which greatest bulge radius is at least 5% greater than an average radius of the substantially cylindrical tubular portions proximally and distally adjacent the bulge,
the struts of the second subset define tip portions, and
the number of the tip portions of the struts of the second subset that define the bulge is at least 50% greater than the average number of circumferential cells in the two circumferentially-continuous rings proximally and distally adjacent the bulge.

2. The apparatus according to claim 1, wherein the number of the tip portions of the struts of the second subset that define the bulge equals between 175% and 250% of the average number of circumferential cells.

3. The apparatus according to claim 2, wherein the number of the tip portions of the struts of the second subset that define the bulge equals 200% of the average number of circumferential cells.

4. The apparatus according to claim 1, wherein the number of the tip portions of the struts of the second subset that define the bulge equals between 12 and 16.

5. The apparatus according to claim 1, wherein the bulge completely circumferentially encircles the stent-graft.

6. The apparatus according to claim 1, wherein the tip portions are directly sutured to the fluid flow guide.

7. The apparatus according to claim 1, wherein the tip portions are shaped so as to define respective atraumatic features.

8. The apparatus according to claim 1, the fluid flow guide is arranged such that all of the fabric defines the lumen when the stent-graft assumes the radially-expanded state.

9. The apparatus according to claim 1, wherein the circumferentially-continuous rings undulate, such that the circumferential cells are shaped so as to define respective peaks and troughs.

10. The apparatus according to claim 9, wherein the peaks and troughs are substantially semicircular, have a first average radius of curvature, and are shaped so as to define respective strain-distribution features having a second average radius of curvature equal to between 10% and 50% of the first average radius of curvature.

11. The apparatus according to claim 1, wherein an axial length of the bulge equals between 10% and 40% of a difference between (a) the greatest bulge radius and (b) the average radius of the substantially cylindrical tubular portions proximally and distally adjacent the bulge.

12. The apparatus according to claim 1, wherein the struts of the first and the second subsets are arranged and shaped such that the struts of the first subset apply a radially-outward force that is at least 20% greater than a radially-outward applied by the struts of the second subset.

13. The apparatus according to claim 12, wherein the struts of the first and the second subsets are arranged and shaped such that the struts of the first subset apply the radially-outward force that is at least 40% greater than the radially-outward applied by the struts of the second subset.

14. The apparatus according to claim 1, wherein the circumferential cells are W-shaped.

15. The apparatus according to claim 1, wherein the struts of the first and the second subsets are superelastic.

16. The apparatus according to claim 1, wherein the circumferential cells are diamond-shaped.

17. The apparatus according to any one of claims 1-16,
wherein the bulge is a first bulge,
wherein the fluid flow guide is fixed to the first subset of the struts, the second subset of the struts, and a third subset of the struts so as to define the lumen, wherein the first, the second, and the third subsets do not include any common struts, and
wherein the struts of the third subset are elastic such that, when the stent-graft assumes the radially-expanded state:
the struts of the third subset cause the fluid flow guide to define a second bulge having a greatest bulge radius from the central longitudinal axis, which greatest bulge radius is at least 5% greater than an average radius of the substantially cylindrical tubular portions proximally and distally adjacent the second bulge,
the struts of the third subset define tip portions, and
the number of the tip portions of the struts of the third subset that define the second bulge is at least 50% greater than the average number of circumferential cells in the two circumferentially-continuous rings proximally and distally adjacent the second bulge.

18. The apparatus according to any one of claims 1-16, wherein at least a first one of the struts of the second subset is structurally integral with at least a second one of the struts of the first subset.

19. The apparatus according to claim 18,
wherein all of the struts of the second subset are structurally integral with at least one of the struts of the first subset,
wherein none of the struts of the second subset is directly connected to any of the other struts of the second subset, and
wherein none of the struts of the second subset is indirectly connected to any of the other struts of the second subset by any struts other than the struts of the first subset.

20. The apparatus according to claim 18, wherein, for at least a first one of the tip portions of first one of the struts of the second subset, an angle is defined by (a) the central longitudinal axis and (b) a line defined by (i) the first one of the tip portions and (ii) a junction between the first one of the struts of the second subset and the second one of the struts of the first subset, and the angle is between 5 and 60 degrees.

21. The apparatus according to claim 20, wherein the angle is between 10 and 30 degrees.

22. The apparatus according to any one of claims 1-16,
wherein the stent-graft further comprises sutures that secure the struts of the first and the second subsets to the fluid flow guide, and
wherein, for at least a first one of the tip portions of the first one of the struts of the second subset:
the first one of the struts of the second subset has a length measured along the first one of the struts between (a) the first tip portion and (b) a junction between the first one of the struts of the second subset and the second one of the struts of the first subset,
a far half of the first one of the struts extends from the first tip portion along 50% of the length of the first one of the struts of the second subset, and
none of the sutures are disposed along at least 50% of the far half of the first one of the struts of the second subset.

23. The apparatus according to any one of claims 1-16,
wherein the struts of the first subset are arranged in a plurality of undulating circumferentially-continuous rings having alternating peaks and troughs, and
wherein a first plurality of the struts of the second subset originate in a proximal half of one of the undulating circumferentially-continuous rings, and a second plurality of the struts of the second subset originate in a distal half of the one of the undulating circumferentially-continuous rings.

24. The apparatus according to claim 23, wherein the first plurality of the struts of the second subset originate in a proximal 20% of an axial height of the one of the undulating circumferentially-continuous rings.

25. The apparatus according to claim 24, wherein the first plurality of the struts of the second subset originate at proximal-most sites of the one of the undulating circumferentially-continuous rings.

26. The apparatus according to claim 24, wherein the second plurality of the struts of the second subset originate in a distal 20% of the axial height of the one of the undulating circumferentially-continuous rings.

27. The apparatus according to claim 26,
wherein the first plurality of the struts of the second subset originate at proximal-most sites of the one of the undulating circumferentially-continuous rings, and
wherein the second plurality of the struts of the second subset originate at distal-most sites of the one of the undulating circumferentially-continuous rings.

28. The apparatus according to claim 23, wherein a first plurality of the struts of the second subset originate at two or more axially different locations of one of the undulating circumferentially-continuous rings.

29. The apparatus according to any one of claims 1-16, wherein at least one of the struts of the second subset is not structurally integral with any of the struts of the first subset.

30. The apparatus according to any one of claims 1-16,
wherein the fluid flow guide is fixed to the first subset of the struts, the second subset of the struts, and a third subset of the struts so as to define the lumen, wherein the first, the second, and the third subsets do not include any common struts, and
wherein the struts of the third subset are elastic such that, when the stent-graft assumes the radially-expanded state, the struts of the third subset cause the fluid flow guide to define a flared axial portion that extends to one end of the fluid flow guide, the flared axial portion having (a) a greatest flared radius from the central longitudinal axis, which greatest flared radius is at least 5% greater than a radius of the substantially cylindrical tubular portion axially adjacent the flared axial portion, and (b) an axial length equal to between 5% and 20% of a difference between (i) the greatest flared radius and (ii) the radius of the substantially cylindrical tubular portion axially adjacent the flared axial portion.

31. The apparatus according to claim 30,
wherein the flared axial portion is a first flared axial portion, and the one end of the fluid flow guide is a first end of the fluid flow guide, and
wherein the struts of the third subset are elastic such that, when the stent-graft assumes the radially-expanded state, the struts of the third subset cause the fluid flow guide to additionally define a second flared axial portion that extends to a second end of the fluid flow guide, the second flared axial portion having (a) a greatest flared radius from the central longitudinal axis, which greatest flared radius is at least 5% greater than an average radius of the substantially cylindrical tubular portion axially adjacent the second flared axial portion, and (b) an axial length equal to between 5% and 20% of a difference between (i) the greatest flared radius and (ii) the radius of the substantially cylindrical tubular portion axially adjacent the second flared axial portion.

32. The apparatus according to any one of claims 1-16,
wherein the stent-graft further comprises sutures that secure the struts of the first and the second subsets to the fluid flow guide, and
wherein at least 80% of a surface area of the struts of the first subset is within 3 mm of at least one of the sutures that secure the struts of the first subset to the fluid flow guide.

33. The apparatus according to claim 32, wherein no more than 50% of the surface area of the struts of the second subset is within 3 mm of at least one of the sutures that secure the struts of the second subset to the fluid flow guide.

34. The apparatus according to any one of claims 1-16, wherein respective circumferences of all substantially cylindrical tubular portions of the fluid flow guide vary by less than 10%.

35. The apparatus according to any one of claims 1-16, wherein a circumference of a first one of the substantially cylindrical tubular portions is at least 10% greater than a circumference of a second one of the substantially cylindrical tubular portions.

36. The apparatus according to any one of claims 1-16, wherein when the endovascular stent-graft is removably disposed in the delivery catheter in the radially-compressed state, the struts of the first subset do not coincide with the struts of the second subset.

37. The apparatus according to any one of claims 1-16,
wherein the stent-graft is a main stent-graft,
wherein the fluid flow guide is not shaped so as to define any fenestrations or scallops, and
wherein the apparatus further comprises one or more branching stent-grafts.

38. The apparatus according to any one of claims 1-16,
wherein the stent-graft is a main stent-graft,
wherein the fluid flow guide is not shaped so as to define one or more openings selected from the group of openings consisting of: fenestrations, scallops, and fenestrations and scallops, and
wherein the apparatus further comprises a number of branching stent-grafts, the number greater than a number of the openings.

39. Apparatus for use with a delivery catheter, the apparatus comprising an endovascular stent-graft, which is configured to initially be positioned in the delivery catheter in a radially-compressed state, and to assume a radially-expanded state upon being deployed from the delivery catheter, and which comprises:
struts; and
a fluid flow guide, which comprises at least one biologically-compatible substantially blood-impervious fabric, and which is fixed to first and second subsets of the struts, wherein the first and the second subsets do not include any common struts,
wherein the struts of the first and the second subsets are elastic such that, when the stent-graft assumes the radially-expanded state:
the fluid flow guide defines a lumen having a central longitudinal axis,
the struts of the first subset are arranged as a plurality of circumferential cells in circumferentially-continuous rings, which cause the fluid flow guide to define a plurality of substantially cylindrical tubular portions,
the struts of the second subset are arranged as a plurality of bulge-inducing units that cause the fluid flow guide to define a bulge having a greatest bulge radius from the central longitudinal axis, which greatest bulge radius is at least 5% greater than an average radius of the substantially cylindrical tubular portions proximally and distally adjacent the bulge, and
an average circumferential width of the bulge-inducing units is no more than 25% of an average circumferential width of the circumferential cells in the two circumferentially-continuous rings proximally and distally adjacent the bulge.

40. A method comprising:
advancing, into a main artery of a subject, an endovascular stent-graft, which is removably disposed in a delivery catheter in a radially-compressed delivery state, and comprises (a) a main stent-graft, which comprises (i) elastic struts and (ii) a fluid flow guide, which comprises at least one biologically-compatible substantially blood-impervious fabric, and which is fixed to first and second subsets of the struts, wherein the first and the second subsets do not include any common struts;
deploying the endovascular stent-graft from the delivery catheter such that the endovascular stent-graft assumes a radially-expanded state in which (a) the fluid flow guide defines a lumen having a central longitudinal axis, (b) the struts of the first subset are arranged as a plurality of circumferential cells in circumferentially-continuous rings, which cause the fluid flow guide to define a plurality of substantially cylindrical tubular portions, (c) the struts of the second subset cause the fluid flow guide to define a bulge having a greatest bulge radius from the central longitudinal axis, which greatest bulge radius is at least 5% greater than an average radius of the substantially cylindrical tubular portions proximally and distally adjacent the bulge, (d) the struts of the second subset define tip portions, and (e) the number of the tip portions of the struts of the second subset that define the bulge is at least 50% greater than the average number of circumferential cells in the two circumferentially-continuous rings proximally and distally adjacent the bulge; and
deploying one or more branching stent-grafts partially alongside the main stent-graft and partially in respective branching arteries that branch from the main artery, such that portions of the branching stent-grafts contact the bulge.
